(19) United States
(12) Patent Application Publication
(10) Pub. No.: US 2026/0049323 A1
(43) Pub. Date: Feb. 19, 2026

(54) EXPRESSION SYSTEMS FOR PHOSPHATASES

(71) Applicant: INTERNATIONAL N&H DENMARK APS, Lyngby (DK)

(72) Inventors: Cristina Bongiorni, Fremont, CA (US); Dmitri V Vaviline, Redwood City, CA (US); Andrew Prior, Menlo Park, CA (US); Lilia Maria Babe, Emerald Hills, CA (US); Amy Deming Liu, Sunnyvale, CA (US)

(21) Appl. No.: 19/103,587

(22) PCT Filed: Aug. 15, 2023
(86) PCT No.: PCT/US2023/072196

§ 371 Date: Feb. 13, 2025

Related U.S. Application Data
(60) Provisional application No. 63/371,616, filed on Aug. 16, 2022.

(51) Int. Cl. C12N 15/75; C12N 9/14

(52) U.S. Cl. CPC C12N 15/75; C12N 9/14; C12Y 306/01005

(57) ABSTRACT

Provided herein, inter alia, are compositions and methods for expressing recombinant phosphatases containing numerous disulfide bonds in host cells.

### CROSS REFERENCE TO RELATED APPLICATIONS

This application claims priority to U.S. Provisional Patent Application No. 63/371,616, filed Aug. 16, 2022, the disclosure of which is herein incorporated by reference in its entirety.

### INCORPORATION BY REFERENCE OF SEQUENCE LISTING

The present application is being filed along with a Sequence Listing in electronic format. The Sequence Listing is provided as a file entitled NB42134-WO-PCT.xml, created 8 Aug. 2023 which is 73,483 bytes in size. The information in the electronic format of the Sequence Listing is incorporated by reference in its entirety.

### FIELD OF THE INVENTION

Provided herein, inter alia, are compositions and methods for expressing phosphatases.

### BACKGROUND

The gram-positive eubacterium Bacillus subtilis is well known for its high capacity to secrete proteins, both in its soil-based natural habitat and in biotechnological applications for the production of recombinant proteins (Darmon et al., Appl. Environ. Microbiol, 2006, 72(11): 6876-85).

Most of the polypeptides secreted by B. subtilis are believed to be exported in an unfolded form from the cytoplasm and have to fold efficiently after membrane translocation. However, expression and secretion of polypeptides containing multiple disulfide bonds can induce a secretion stress response in B. subtilis host cells thereby lowering overall expression and secretion (Darmon et al., Applied and Environmental Microbiology, 2006, 72(11): 6876-85). Other studies have found similar difficulties with the expression of polypeptides containing disulfide bonds in B. subtilis (Westers et al., Biochimica et Biophysica Acta, 2004, 1694:299-310).

In Bacilli and other Gram-positives, little is known about disulfide bond formation and isomerization. In these organisms the presence of thiol-disulfide oxidoreductases was questioned for a long time, because their secreted proteins were not found to contain disulfide bridges and the formation of disulfide bonds in secreted heterologous proteins occurred very inefficiently (Westers et al., Biochimica et Biophysica Acta, 2004, 1694:299-310; Bolhuis et al., Appl. Environ. Microbiol., 1999, 65:2934-41).

Consequently, there is a need for B. subtilis host cell expression systems that can produce correctly folded and functional disulfide bond-containing polypeptides in commercially relevant quantities.

The subject matter disclosed herein addresses these needs and provides additional benefits as well.

### SUMMARY

Provided herein, inter alia, are compositions and methods for expressing recombinant phosphatases (such as acid phosphatases and/or apyrases) containing numerous disulfide bonds in Gram-positive host cells (such as B. subtilis host cells).

Accordingly, in some aspects, provided herein are a recombinant Gram-positive host cell comprising a nucleic acid at least about 60% identical to SEQ ID NO:9 or SEQ ID NO:37 or a fragment thereof, wherein the host cell comprises a deletion of one or more endogenous genes encoding a protease. In some embodiments, the nucleic acid encodes a polypeptide at least about 80% identical to SEQ ID NO:10 or SEQ ID NO:25 or a functional fragment thereof. In some embodiments, the nucleic acid encodes a polypeptide at least about 80% identical to SEQ ID NO: 26, SEQ ID NO:27 or SEQ ID NO:28 or a functional fragment thereof. In some embodiments of any of the embodiments disclosed herein, the host cell has one, two, three, four, five, six, seven, eight, or nine endogenous genes encoding a protease deleted. In some embodiments, the host cell comprises nine endogenous genes encoding a protease deleted. In some embodiments of any of the embodiments disclosed herein, the one or more endogenous genes encoding a protease comprise one or more of aprE, nprE, epr, ispA, bpr, mpr, vpr, wprA and/or nprB. In some embodiments of any of the embodiments disclosed herein, the polypeptide comprises one or more disulfide bonds. In some embodiments, the polypeptide comprises seven disulfide bonds. In some embodiments of any of the embodiments disclosed herein, the nucleic acid is expressed on an extrachromosomal vector. In some embodiments of any of the embodiments disclosed herein, the nucleic acid is integrated into the genome of the host cell. In some embodiments of any of the embodiments disclosed herein, the host cell is a Bacillus spp. In some embodiments, the Bacillus spp. is B. subtilis. In some embodiments, the polypeptide is secreted from the host cell.

In further aspects, provided herein is a method for producing a recombinant protein comprising culturing any of the host cells disclosed herein in a suitable media. In some embodiments, the method further comprises purifying the recombinant protein.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 is a line graph depicting production of G. xiamenensis CRC22110 apyrase in Bacillus subtilis AP186 and the negative control strain AP128 in a time course experiment.

FIG. 2 is a line graph depicting a comparison of ATPase activity from samples of cultivation of E. coli host strains AP391 (encoding CRC22110 gene) and AP390 (lacking gene of interest.

### DETAILED DESCRIPTION

This invention is based, at least in part, on the inventors' surprising discovery that expressing a phosphatase protein in a Gram-positive host cell (such as a B. subtilis host cell) can result in useful yields of functional enzyme. The functional protein production occurs in spite of the fact that the phosphatase protein possesses extensive disulfide bonding. As discussed in the Background section, the available literature suggests that disulfide bond formation can induce a secretion stress response in Gram-positive expression hosts (such as, B. subtilis), thereby lowering overall expression and secretion.

### I. Definitions

The term “acid phosphatase” (EC 3.1.3.2) (also known as acid phosphomonoesterase, phosphomonoesterase, glycerophosphatase, acid monophosphatase, acid phosphohydrolase, acid phosphomonoester hydrolase, uteroferrin, acid nucleoside diphosphate phosphatase, acid phosphatase (class A) or orthophosphoric-monoester phosphohydrolase (acid optimum)) is used to mean an enzyme having a pH optimum for mediating hydrolysis of a phosphate ester bond in a substrate at a pH less than about pH 6.5, such as less than about pH 4.0. Acid phosphatases free attached phosphoryl groups from other molecules during digestion. It can be further classified as a phosphomonoesterase. Acid phosphatase is stored in lysosomes and functions when these fuse with endosomes, which are acidified while they function; therefore, it has an acid pH optimum. This enzyme is present in many animal and plant species. In some embodiments, the acid phosphatase used in the compositions and methods disclosed herein is not derived from Shigella. In some embodiments, the acid phosphatase used in the compositions and methods disclosed herein is not derived from a mammal. In some embodiments, the enzyme expressed in any of the host cells disclosed herein (for example a B. subtilis host cell) is an acid phosphatase.

As used herein, the “GDA1_CD39 superfamily refers to enzymes comprised of nucleoside triphosphate diphosphohydolases (NTPDases) with common motifs in their protein sequences. The family is named after two proteins: the yeast GDPase (GDA1) and a lymphoid cell activation antigen, CD39. These proteins, in some embodiments, are cell-surface enzymes that hydrolyze a range of NTPs, including extracellular ATP. Non-limiting examples include ecto-ATPases, apyrases, CD39s, and ecto-ATP/Dases (Knowles, 2011, Purinergic Signalling volume 7, pages 21-45, incorporated by reference herein). In some embodiments, the enzyme expressed in any of the host cells disclosed herein (for example a B. subtilis host cell) is a member of the GDA1_CD39 superfamily.

The term “apyrase,” as used herein, refers to one or more of a calcium-activated enzyme (i.e. proteins belonging to class EC. 3.6.1.5) which possesses ATP-diphosphohydrolase activity and catalyzes the hydrolysis of the gamma phosphate from ATP, and catalyzes the hydrolysis of the beta phosphate from ADP. Apyrases are found in all eukaryotes and some prokaryotic organisms, indicating a preserved role for these enzymes across species. They possess a distinct phosphohydrolase activity, nucleotide substrate specificity, divalent cation requirement, and sensitivity to inhibitors. (See, Plesner, Int. Rev. Cytol., 158:141 (1995), and Handa & Guidotti, Biochem. Biophys. Res. Commun., 218(3): 916 (1996)). In mammals, apyrase is believed to function primarily as an extracellular hydrolase specific for ATP and ADP, which function is important in the inactivation of synaptic ATP molecules following nerve stimulation. (See, Todorov et al., Nature, 387(6628): 76 (1997)). Apyrase in mammals is also believed to be important in the inhibition of ADP-induced platelet aggregation. (See, Marcus et al., J. Clin. Invest., 99(6): 1351 (1997)). Recombinant apyrase is commercially available from New England Biolabs. In some embodiments, the apyrase used in the compositions and methods disclosed herein is not derived from potato. In other embodiments, the apyrase used in the compositions and methods disclosed herein is not derived from a mammal. In some embodiments, the enzyme expressed in any of the host cells disclosed herein (for example a B. subtilis host cell) is an apyrase.

As used herein, “microorganism” or “microbe” refers to a bacterium, a fungus, a virus, a protozoan, and other microbes or microscopic organisms.

The terms “protein” and “polypeptide” refer to compounds comprising amino acids joined via peptide bonds and may be used interchangeably. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Amino acid positions in a given polypeptide sequence can be named by the one letter code for the amino acid, followed by a position number. For example, a glycine (G) at position 87 is represented as “G087” or “G87.”

As used herein, where “amino acid sequence” is recited it refers to an amino acid sequence of a protein or peptide molecule. An “amino acid sequence” can be deduced from the nucleic acid sequence encoding the protein. However, terms such as “polypeptide” or “protein” are not meant to limit the amino acid sequence to the deduced amino acid sequence but can include posttranslational modifications of the deduced amino acid sequences, such as amino acid deletions, additions, and modifications such as glycosylations and addition of lipid moieties. Also, the use of non-natural amino acids, such as D-amino acids to improve stability or pharmacokinetic behavior falls within the scope of the term “amino acid sequence”, unless indicated otherwise.

The term “mature” form of a protein, polypeptide, or peptide refers to the functional form of the protein, polypeptide, or enzyme without the signal peptide sequence and propeptide sequence.

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g, proteins, amino acids, or nucleic acid sequences) that is found in nature. Conversely, the term “non-naturally occurring” refers to anything that is not found in nature (e.g, recombinant/engineered nucleic acids and protein sequences produced in the laboratory or modification of the wild-type sequence).

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide.

As used herein, the terms “recombinant” or “non-natural” refer to an organism, microorganism, cell, nucleic acid molecule, vector and the like that has at least one engineered genetic alteration, or has been modified by the introduction of a heterologous nucleic acid molecule; or refer to a cell (e.g., a Gram positive cell) that has been altered such that the expression of a heterologous nucleic acid molecule or an endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules (e.g., polynucleotide constructs) that are not found in identical or homologous form within a native (wild-type) cell, or may provide an altered expression pattern of endogenous genes, such as being over-expressed, under-expressed, minimally expressed, or not expressed at all.

“Recombination”, “recombining” or generating a “recombined” nucleic acid is generally the assembly of two or more nucleic acid fragments wherein the assembly gives rise to a chimeric DNA sequence that would not otherwise be found in the genome.

The term “derived” encompasses the terms “originated”, “obtained,” “obtainable,” and “created,” and generally indicates that one specified material or composition finds its origin in another specified material or composition, or has features that can be described with reference to the another specified material or composition. For example, recombinant Gram-positive bacterial cells of the disclosure may be derived/obtained from any known Gram-positive bacterial strains (e.g., B. subtilis 168 strain, etc.).

As used herein, an “endogenous gene” refers to a gene in its natural location in the genome of an organism.

As used herein, a “heterologous” gene, a “non-endogenous” gene, or a “foreign” gene refer to a gene (or gene coding sequence; CDS)/open reading frame; ORF) not normally found in the host organism, but that is introduced into the host organism by gene transfer. The term “foreign” gene(s) comprise native genes (or ORF's) inserted into a non-native organism and/or chimeric genes inserted into a native or nonnative organism.

As used herein, a “heterologous control sequence”, refers to a gene expression control sequence (e.g., promoters, enhancers, terminators, etc.) which does not function in nature to regulate (control) the expression of the gene of interest. Generally, heterologous nucleic acids are not endogenous (native) to the cell, or a part of the genome in which they are present, and have been added to the cell, by infection, transfection, transduction, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase during translocation.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, “nucleic acid” refers to a nucleotide or polynucleotide sequence, and fragments or portions thereof, as well as to DNA, cDNA, and RNA of genomic or synthetic origin, which may be double stranded or single-stranded, whether representing the sense or antisense strand. It will be understood that as a result of the degeneracy of the genetic code, a multitude of nucleotide sequences may encode a given protein. It is understood that the polynucleotides (or nucleic acid molecules) described herein include “genes”, “vectors” and “plasmids”. Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located (3′) downstream to a promoter sequence. Promoters may be derived in their entirety from a native gene or be composed of different elements derived from different promoters found in nature, or even comprise synthetic nucleic acid segments.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation. Thus, a nucleic acid is “operably linked” when it is placed into a functional relationship with another nucleic acid sequence. For example, DNA encoding a secretory leader (e.g., secretory signal sequence) is operably linked to DNA for a polypeptide if it is expressed as a pre-protein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in a desired Gram-positive host cell. For example, in certain embodiments, the present disclosure is directed to a polynucleotide comprising an upstream (5′) promoter (or 5′ promoter region, or tandem 5′ promoters and the like) functional in a Gram-positive cell, wherein the promoter region is operably linked to a nucleic acid sequence (e.g., an ORF) encoding an acid phosphatase protein.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, transcription leader sequences, RNA processing site, effector binding site and stem-loop structures.

As used herein, the terms “modification” and “genetic modification” are used interchangeably and include, but are not limited to: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein (such that a functional protein is not made), internal deletions, insertions disrupting the coding sequence, mutations removing the operable link between a native promoter required for transcription and the open reading frame, and the like.

As used herein, the term “introducing”, as used in phrases such as “introducing into a bacterial cell” or “introducing into a bacterial cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein “an incoming sequence” refers to a DNA sequence that is introduced into the Gram-positive host cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wildtype gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” or “homology arm” refers to a nucleic acid sequence, which is homologous to a sequence in the Gram-positive bacterial cell's chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, downregulated and the like, according to the invention. These sequences direct where in the Gram-positive bacterial cell's chromosome a DNA construct is integrated and directs what part of the Gram-positive bacterial cell's chromosome is replaced by the incoming sequence. While not meant to limit the present disclosure, a homology box may include about between 1 base pair (bp) to 200 kilobases (kb). In some embodiments, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., ampR, phleoR, specR, kanR, eryR, tetR, cmpR and neoR. Other markers useful in accordance with the invention include, but are not limited to auxotrophic markers, such as serine, lysine, tryptophan; and detection markers, such as β-galactosidase.

As defined herein, a host cell “genome”, a Gram-positive bacterial (host) cell “genome”, a Bacillus sp. (host) cell “genome” and the like, include chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. In some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophages, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously) or can integrate into a chromosome of a host organism.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental Bacillus sp. (host) cell is modified (e.g., transformed) by introducing therein one or more “targeting vectors”.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in some embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

As used herein, a “host cell” refers to a cell that has the capacity to act as a host or expression vehicle for a newly introduced DNA sequence. In certain aspects, a host cell of the disclosure is a Gram-positive bacterial cell/strain. As appreciated by one skilled in the art, many Gram-positive host strains are generally recognized as safe (GRAS) per US FDA guidelines, and as such, Gram-positive host cells are particularly useful protein production hosts relative to Gram-negative hosts (e.g., E. coli expression systems), which require additional costly processing steps to remove endotoxins (e.g., LPS).

As used herein, the B. subtilis strain named “CBS12” was constructed for expression and secretion of an apyrase derived from Gallaecimonas xiamenensis (SEQ ID NO: 10 or SEQ ID NO: 25).

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “protein preparation” is any material, typically a solution, generally aqueous, comprising one or more proteins.

As used herein, the terms “broth”, “cultivation broth”, “fermentation broth” and/or “whole fermentation broth” may be used interchangeably, and refer to a preparation produced by cellular fermentation that undergoes no processing steps after the fermentation is complete. For example, whole fermentation broths are typically produced when microbial cultures are grown to saturation, incubated under carbon-limiting conditions to allow protein synthesis (e.g., expression of proteins by host cells; and optionally, secretion of the proteins into cell culture medium). Typically, the whole fermentation broth is unfractionated and comprises spent cell culture medium, metabolites, extracellular polypeptides, and microbial cells.

As used herein, the phrase “treated broth” refers to broth that has been conditioned by making changes to the chemical composition and/or physical properties of the broth. Broth “conditioning” may include one or more treatments such as cell lysis, pH modification, heating, cooling, addition of chemicals (e.g., calcium, salt(s), flocculant(s), reducing agent(s), enzyme activator(s), enzyme inhibitor(s), and/or surfactant(s)), mixing, and/or timed hold (e.g., 0.5 to 200 hours) of the broth without further treatment.

As used herein, a “cell lysis” process includes any cell lysis technique known in the art, including but not limited to, enzymatic treatments (e.g., lysozyme, proteinase K treatments), chemical means (e.g., ionic liquids), physical means (e.g., French pressing, ultrasonic), simply holding culture without feeds, and the like.

The terms “recovery,” “recovered,” and “recovering” as used herein refer to at least partial separation of a protein from one or more components of a microbial broth and/or at least partial separation from one or more solvents in the broth (e.g., water or ethanol).

In certain aspects, broths in which host cells have been fermented for the production of phosphatase proteins, with or without broth treatment, are clarified. As used herein, a “clarified” broth means a broth which has been subjected to at least one clarification process to remove cell debris and/or other insoluble components. Clarification processes, as understood in the art include, but are not limited to, centrifugation techniques, cross-flow membrane filtration techniques, solid/liquid filtration techniques, and the like.

“Cell debris” refers to cell walls and other insoluble components that are released or formed after disruption of the cell membrane (e.g., after performing a cell lysis process).

In certain aspects, separation of solvents, as understood in the art include, but are not limited to ultrafiltration, evaporation, spray drying, freezer drying. The obtained solution is referred to as “clarified broth concentrate”, “UF concentrate”, or “ultrafiltrate concentrate”.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

### A. Enzymes

Provided herein are isolated proteins having phosphatase activity. A phosphatase is an enzyme that dephosphorylates its substrates; i.e., it hydrolyses phosphoric acid monoesters into a phosphate ion and a molecule with a free hydroxyl group. Phosphatases include apyrases. This action is directly opposite to that of phosphorylases and kinases, which attach phosphate groups to their substrates by using energetic molecules like ATP.

The phosphatases for use in the compositions and methods disclosed herein are active at least from about pH 3 to pH 9 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9) or from pH 3.5 to 5.5 or from pH 3.5 to 7, or from pH 5 to 7, or from pH 3.5 to 9, or from pH 5 to 9. In some embodiments, the enzyme comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity), or at least 35% or at least 60% sequence identity (such as any of about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of SEQ ID NO: 10 or SEQ ID NO:25 (CRC22110). In some embodiments, the enzyme comprises the amino acid sequence of SEQ ID NO: 10 or SEQ ID NO: 25 (CRC22110).

In some embodiments, the enzyme comprises a nucleic acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity), or at least 35% or at least 60% sequence identity (such as any of about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length nucleic sequence of SEQ ID NO: 9 or SEQ ID NO:37. In some embodiments, the enzyme comprises a nucleic acid sequence having at least 60% sequence identity to SEQ ID NO: 9 or SEQ ID NO:37. In some embodiments, the enzyme comprises the nucleic acid sequence of SEQ ID NO: 9 or SEQ ID NO:37.

In some of any embodiments, the enzyme is a polypeptide with one or more modifications. In some embodiments, the modification comprises a deletion, insertion or substitution. In some embodiments, the modification comprises a truncation. In some embodiments, the truncation is of the N-terminus. In some embodiments, the truncation is of the C-terminus. In some embodiments, the modification comprises an insertion. In some embodiments, the insertion is on the N-terminus. In some embodiments, the insertion is on the C-terminus. In some embodiments, any of the polypeptides provided herein further contains a histidine (His) tag.

In some embodiments, the enzyme comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity), or at least 35% or at least 60% sequence identity (such as any of about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) with the full length amino acid sequence of SEQ ID NO:26, SEQ ID NO:27 or SEQ ID NO:28. In some embodiments, the enzyme comprises the amino acid sequence of SEQ ID NO:26, SEQ ID NO:27 or SEQ ID NO:28.

In further embodiments, phosphatases for use in the compositions and methods disclosed herein 1) are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9) or from pH 3.5 to 5.5 or from pH 3.5 to 7 or from pH 5 to 7), or at least from about pH 3 to pH 9 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7, or from pH 3.5 to 9, or from pH 5 to 9) 2) comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) or at least 35% or at least 60% sequence identity (such as any of about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity), with the full length amino acid sequence of one or more of SEQ ID NO: 10 or SEQ ID NO:25; and 3) have amino acid residues S204, F205, L206, G207, L208, and G209 at positions corresponding to the numbering of SEQ ID NO:10.

In further embodiments, phosphatases for use in the compositions and methods disclosed herein 1) are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9) or from pH 3.5 to 5.5 or from pH 3.5 to 7 or from pH 5 to 7), or at least from about pH 3 to pH 9 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7, or from pH 3.5 to 9, or from pH 5 to 9) 2) comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) or at least 35% or at least 60% sequence identity (such as any of about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity), with the full length amino acid sequence of one or more of SEQ ID NO: 10 or SEQ ID NO:25; and 3) have amino acid residues S204, F205, L206, G207, L208, and G209 at positions corresponding to the numbering of SEQ ID NO:25.

In further embodiments, phosphatases for use in the compositions and methods disclosed herein 1) are active at least from about pH 3 to pH 7 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9) or from pH 3.5 to 5.5 or from pH 3.5 to 7 or from pH 5 to 7), or at least from about pH 3 to pH 9 (such as any of about pH 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7) or from pH 3.5 to 5.5 or from pH 3.5 to 7 or from pH 5 to 7, or from pH 3.5 to 9, or from pH 5 to 9) 2) comprises an amino acid sequence having at least 40% or at least 60% sequence identity (such as any of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) or at least 35% or at least 60% sequence identity (such as any of about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity), with the full length amino acid sequence of one or more of SEQ ID NO:26, SEQ ID NO:27 or SEQ ID NO: 28; and 3) have amino acid residues S204, F205, L206, G207, L208, and G209 at positions

### B. Host Cells

As briefly set forth above, and further described in Example section below, provided herein are designed, constructed and evaluated exemplary Gram-positive bacterial cells capable of expressing heterologous proteins. For example, polynucleotides (expression cassettes) encoding a phosphatase protein (CRC22110; SEQ ID NO: 10 or SEQ ID NO:25) or a truncated variant (SEQ ID NO:26, SEQ ID NO:27 or SEQ ID NO:28) can be introduced into Bacillus cells, and evaluated in large scale bioreactors. More specifically, expression cassettes encoding a G. xiamenensis phosphatase (CRC22110; SEQ ID NO:10 or SEQ ID NO:25) or a truncated variant (SEQ ID NO:26, SEQ ID NO:27 or SEQ ID NO:28) were constructed for intracellular expression, or secreted expression of the phosphatase in Bacillus cells. In some embodiments, expression cassettes encoding a G. xiamenensis phosphatase (CRC22110; SEQ ID NO:10 or SEQ ID NO:25) or a truncated variant (SEQ ID NO:26, SEQ ID NO:27 or SEQ ID NO:28) were constructed for secreted expression of the phosphatase in Bacillus cells. In some embodiments, the polypeptide is secreted from the host cell. In some embodiments, the polypeptide is secreted from the host cell into medium. Thus, certain embodiments of the disclosure provide recombinant Gram-positive bacterial cells expressing one or more heterologous nucleic acids (polynucleotides) encoding phosphatase proteins.

In certain embodiments, a recombinant Gram-positive bacterial cell expresses a heterologous polynucleotide encoding native phosphatase protein, or a functional variant derived from the native phosphatase protein. In certain aspects, heterologous polynucleotides encoding phosphatase proteins are expression cassettes introduced into the recombinant cell. In certain embodiments, at least one expression cassette is introduced in the Gram-positive bacterial cell. In other embodiments, at least two expression cassettes are introduced in the Gram-positive bacterial cell. Thus, in certain aspects Gram-positive host cells of the disclosure comprise one or more phosphatase expression cassette introduced therein, wherein the host cells express the phosphatase when cultivated under suitable conditions.

In certain aspects, a Gram-positive bacterial cell includes the classes Bacilli, Clostridia and Mollicutes (e.g., including Lactobacillies with the families Acrococcaceae, Carnobacteriaceae, Enterococcaceae, Lactobacilloac, Leuconostocaceae, Oscillospiraceae, Streptococcaceae and the Bacillales with the families Alicyclobacellaceae, Bacillaceae, Caryophanaceae, Listeriaceae, Paenibacillaceae, Planococcaceae, Sporolactobacilloae, Staphylococcaceae, Thermoactinomycetaceae, Turicibacteraceae).

In certain embodiments, species of the family Bacillaceae include Alkalibacillus, Amphibacillus, Anoxybacillus, Bacillus, Caldalkalibacillus, Cerasilbacillus, Exiguobacterium, Filobacillus, Geobacillus, Gracilibacillus, Halobacillus, Halolactibacillus, Jeotgalibacillus, Lentibacillus, Marinibacillus, Oceanobacillus, Ornithinibacillus, Paraliobacillus, Paucisalibacillus, Pontibacillus, Pontibacillus, Saccharococcus, Salibacillus, Salinibacillus, Tenuibacillus, Thalassobacillus, Ureibacillus, and Virgibacillus.

In other embodiments, a Bacillus sp. cell includes, but is not limited to, B. acidiceler, B. acidicola, B. acidocaldarius, B. acidoterrestris, B. aeolius, B. aerius, B. aerophilus, B. agaradhaerens. B. agri, B. aidingensis, B. akibai, B. alcalophilus, B. algicola, B. alginolyticus, B. alkalidiazo-trophicus, B. alkalinitrilicus, B. alkalitelluris, B. altitudinis, B. alveayuensis, B. alvei, B. amylolyticus, B. aneurinilyticus, B. aneurinolyticus, B. anthracia, B. aquimaris, B. arenosi, B. arseniciselenatis, B. arsenicoselenatis, B. arsenicus, B. arvi, B. asahii, B. atrophaeus, B. aurantiacus, B. axarquiensis, B. azotofixans, B. azotoformans, B. badius, B. barbaricus, B. bataviensis, B. beijingensis, B. benzoevorans, B. bogoriensis, B. boroniphilus, B. borstelenis, B. butanolivorans, B. carboniphilus, B. cecembensis, B. cellulosilyticus, B. centrosporus, B. chagannorensis, B. chitinolyticus, B. chondroitinus, B. choshinensis, B. cibi, B. circulans,

B. clarkii, B. clausii, B. coagulans, B. coahuilensis, B. cohnii, B. curdianolyticus, B. cycloheptanicus, B. decisifrondis, B. decolorationis, B. dipsosauri, B. drentensis, B. edaphicus, B. ehimensis, B. endophyticus, B. farraginis, B. fastidiosus, B. firmus, B. plexus, B. foraminis, B. fordii, B. formosus, B. fortis, B. fumarioli, B. funiculus, B. fusiformis, B. galactophilus, B. galactosidilyticus, B. gelatini, B. gibsonii, B. ginsengi, B. ginsengihumi, B. globisporus, B. globisporus subsp. globisporus, B. globisporus subsp. marinus, B. glucanolyticus, B. gordonae, B. halmapalus, B. haloalkaliphilus, B. halodenitrificans, B. halodurans, B. halophilus, B. hemicellulosilyticus, B. herbersteinensis, B. horikoshii, B. horti, B. hemi, B. hwajinpoensis, B. idriensis, B. indicus, B. infantis, B. infernus, B. insolitus, B. isabeliae, B. jeotgali, B. kaustophilus, B. kobensis, B. koreensis, B. kribbensis, B krulwichiae, B. laevolacticus, B. larvae, B. laterosporus, B. lautus, B. lehensis, B. lentimorbus, B. lentus, B. litoralis, B. luciferensis, B. macauensis, B. macerans, B. macquariensis, B. macyae, B. malacitensis, B. mannanilyticus, B. marinus, B. marisflavi, B. marismortui, B. massiliensis, B. methanolicus, B. migulanus, B. mojavensis, B. mucilaginosus, B. muralis, B. murimartini, B. mycoides, B. naganoensis, B. nealsonii, B. neidei. B, niabensis, B. niacini, B. novalis, B. odysseyi, B. okhensis, B. okuhidensis, B. oleronius, B. oshimensis, B. pabuli, B. pallidus, B. pallidus (illeg.), B. panaciterrae, B. pantothenticus, B. parabrevis, B. pasteurii, B. patagoniensis, B. peoriae, B. plakortidis, B. pocheonensis, B. polygoni, B. polymyxa, B. popilliae, B. pseudalcaliphilus, B. pseudofirmus, B. pseudomycoides, B. psychrodurans, B. psychrophilus, B. psychrosaccarolyticus, B. psychrotolerans, B. pulvifaciens, B. pycnus, B. qingdaonensis, B. reuszeri, B. runs, B. safensis, B. salarius, B. salexigens, B. saliphilus, B. schlegelii, B. selenatarsenatis, B. selenitrireducens, B. seohaeanensis, B. shackletonii, B. silvestris, B. simplex, B. siralis, B. smithii, B. soli, B. sonorensis, B. sphaericus, B. sporothermodurans, B. stearothermophilus, B. stratosphericus, B. subterraneus, B. subtilis subsp. spizizenii, B. subtilis subsp. subtilis, B. taeanensis, B. tequilensis, B. thermantarcticus, B. thermoaerophilus, B. thermoamylovorans, B. thermoantarcticus, B. thermocatenulatus, B. thermocloacae, B. thermodenitrificans, B. thermoglucosidasius, B. thermoleovorans, B. thermoruber, B. thermosphaericus, B. thiaminolyticus, B. thioparans, B. thuringiensis, B. tusciae, B. validus, B. vallismortis, B. vedderi, B. velezensis, B. vietnamensis, B. vireti, B. vulcani, B. wakoensis and B. weihenstephanensis.

In a particular embodiment, the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. As used herein, the “Bacillus genus” include Bacillus sp. that have been reclassified, including, but not limited to B. stearothermophilus, which is now named “Geobacillus stearothermophilus”.

As generally set forth above and further described below in the Examples, certain embodiments of the disclosure are related to recombinant Gram-positive bacterial cells expressing heterologous phosphatase (including apyrase) proteins, recombinant polynucleotides (e.g. vectors, expression cassettes) encoding heterologous phosphatase proteins particularly suitable for introducing (e.g., transforming) into Gram-positive host cells (i.e., for the expression of heterologous phosphatase) and the like. In other embodiments, Gram-positive host cells of the disclosure are rendered deficient in the production of one or more native (endogenous) proteins. In certain aspects, recombinant host cells of the disclosure comprise deletions or disruptions of one or more endogenous genes encoding one or more proteases native to the recombinant cell. For example, in certain embodiments, recombinant Gram-positive cells rendered deficient in the production of one or more native (endogenous) proteases may be used to mitigate phosphatase degradation (e.g., during fermentation and/or downstream processing of the phosphatase preparation). It is also contemplated herein that recombinant Gram-positive cells rendered deficient in the production of one or more native background proteases, or other problematic (native) background proteins, will facilitate phosphatase downstream recovery and purification (e.g., by reducing undesired host cell background (native) protein contaminants).

Thus, certain embodiments are related to, inter alia, nucleic acids, polynucleotides (e.g., plasmids, vectors, expression cassettes), regulatory elements, and the like, suitable for use in constructing recombinant Gram-positive host cells. Accordingly, as presented in the Examples and generally described herein, recombinant cells of the disclosure may be constructed by one of skill using standard and routine recombinant DNA and molecular cloning techniques well known in the art. Methods for genetically modifying cells include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

In certain embodiments, recombinant (modified) cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby eliminating their expression, or expressing a non-functional (or reduced activity) protein product.

In such methods, the deletion of the gene(s) may be accomplished by homologous recombination using a plasmid that has been constructed to contiguously contain the 5′ and 3′ regions flanking the gene. By way of example, the contiguous 5′ and 3′ regions may be introduced into a Bacillus cell (e.g., on a temperature sensitive plasmid such as pE194) in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers.

Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof.

For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a host cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to, RNA interference (RNAi), small interfering RNA (siRNA), microRNA (miRNA), antisense oligonucleotides, and the like, all of which are well known to the skilled artisan.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions and selecting for mutant cells exhibiting reduced or no expression of the gene.

PCT Publication No. WO2003/083125 (incorporated by reference herein) discloses methods for modifying Bacillus cells, such as the creation of Bacillus deletion strains and DNA constructs using PCR fusion to bypass E. coli. PCT Publication No. WO2002/14490 (incorporated by reference herein) discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli, Bacillus sp.). Indeed, such methods as transformation including protoplast transformation and congression, transduction, and protoplast fusion are known and suited for use in the present disclosure. Methods of transformation are particularly suitable to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Gram-positive bacterial strain by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Gram-positive cells are generally known on one of skill in the art. Promoter sequences of the disclosure are generally chosen so that they are functional in the Gram-positive host cells (e.g., Bacillus cells such as B. licheniformis cells, B. subtilis cells, B. amyloliquefaciens and the like). For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B licheniformis or other related Bacilli. Methods for screening and creating promoter libraries with a range of activities (promoter strength) in Bacillus cells is described in PCT Publication No. WO2002/14490 (incorporated by reference herein).

### C. Enzyme Compositions

Any of the enzymes for use in the methods disclosed herein can be formulated in compositions (for example, pharmaceutical or nutritional compositions). In some embodiments, a herein described protein having phosphatase activity at acid pH ranges according to the invention is useful in treating and/or preventing diseases associated with inflammation in the gut. In one embodiment, the invention provides a composition, preferably a pharmaceutical composition, comprising a protein according to the invention. Said pharmaceutical composition optionally comprises a pharmaceutical acceptable carrier, diluent or excipient.

The composition can be presented in any form, for example as a tablet, as an injectable fluid or as an infusion fluid etc. Moreover, the composition, protein, nucleotide and/or vector according to the invention can be administered via different routes, for example intravenously, rectally, bronchially, or orally. Yet another suitable route of administration is the use of a duodenal drip.

In a one embodiment, the used route of administration is the intravenous route. It is clear for the skilled person, that preferably an effective amount of a protein according to the invention is delivered. As a start point, 1-50,000 U/kg/day can be used. Another suitable route, e.g., for HPP, is the subcutaneous route. If the intravenous route of administration is used, a protein according to the invention can be (at least for a certain amount of time) applied via continuous infusion.

Said composition according to the invention can optionally comprise pharmaceutically acceptable excipients, stabilizers, activators, carriers, permeators, propellants, disinfectants, diluents and preservatives. Suitable excipients are commonly known in the art of pharmaceutical formulation and may be readily found and applied by the skilled artisan, references for instance Remington's Pharmaceutical Sciences, Mace Publishing Company, Philadelphia Pa., 17th ed. 1985.

For oral administration, the protein can, for example, be administered in solid dosage forms, such as capsules, tablets (e.g., with an enteric coating), and powders, or in liquid dosage forms, such as elixirs, syrups, and suspensions. Acid phosphatases and/or apyrases can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable colour, taste, stability, buffering capacity, dispersion or other known desirable features are red iron oxide, silica gel, sodium lauryl sulphate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Enteric coatings arrest the release of the active compound from orally ingestible dosage forms. Depending upon the composition and/or thickness, the enteric coatings are resistant to stomach acid for required periods of time before they begin to disintegrate and permit slow release of acid phosphatase and/or apyrase in the lower stomach, small intestines, or large intestine. Examples of some enteric coatings are disclosed in U.S. Pat. No. 5,225,202 (incorporated by reference). Examples of enteric coatings comprise beeswax and glyceryl monostearate; beeswax, shellac and cellulose, optionally with neutral copolymer of polymethacrylicacid esters; copolymers of methacrylic acid and methacrylic acid methylesters or neutral copolymer of polymethacrylic acid esters containing metallic stearates (for references enteric coatings see: U.S. Pat. Nos. 4,728,512, 4,794,001, 3,835,221, 2,809,918, 5,225,202, 5,026,560, 4,524,060, 5,536,507). Most enteric coating polymers begin to become soluble at pH 5.5 and above, with a maximum solubility rates at pH above 6.5. Enteric coatings may also comprise subcoating and outer coating steps, for instance for pharmaceutical compositions intended for specific delivery in the lower GI tract, i.e. in the colon (pH 6.4 to 7.0, ileum pH 6.6), as opposed to a pH in the upper intestines, in the duodenum of the small intestines the pH ranges 7.7-8 (after pancreatic juices and bile addition). The pH differences in the intestines may be exploited to target the enteric-coated acid phosphatase and/or apyrase composition to a specific area in the gut. It also allows the selection of a specific acid phosphatase and/or apyrase enzyme that is most active at a particular pH in the intestine.

Besides the fact that a protein according to the invention can be incorporated in a pharmaceutical composition such an acid phosphatase and/or apyrase can also be part of a nutritional composition or a nutraceutical.

A protein according to the invention can be added to a nutrient (such as milk) but can also be produced within said nutrient (for instance by molecular engineering). Moreover, tablets and/or capsules can be prepared which are subsequently added to a nutrient or which can be taken directly by a human being.

In yet another aspect, the invention features beverage and food products comprising an acid phosphatase and/or an apyrase effective to treat or prevent gut inflammation in a subject in need thereof. The beverage products can contain from 1 to 10,000, e.g., 1 to 200, 200 to 500, 500 to 1,000, 1,000 to 5,000, or 5,000 to 10,000, units per milliliter. The food products can contain from 1 to 10,000, e.g., 1 to 200, 200 to 500, 500 to 1,000, 1,000 to 5,000, or 5,000 to 10,000, units per gram.

### III. Methods

### A. Cell Culture Methods

In certain embodiments, the present disclosure provides recombinant cells capable of producing proteins of interest (for example, a phosphatase). More particularly, certain embodiments are related genetically modified (recombinant) Gram-positive bacterial cells expressing heterologous phosphatases. Thus, particular embodiments are related to cultivating (fermenting) Gram-positive cells for the production of phosphatase proteins. In general, fermentation methods well known in the art are used to ferment the Gram-positive cells.

In some embodiments, the cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Culturing/fermenting is generally accomplished in a growth medium comprising an aqueous mineral salts medium, organic growth factors, a carbon and energy source material, molecular oxygen, and, of course, a starting inoculum of the microbial host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for most Gram-positive cells the temperature generally will be within the range of about 20° C. to 40° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. Preferences for pH range of microorganisms are dependent on the media employed to some extent, as well as the particular microorganism, and thus change somewhat with change in media as can be readily determined by those skilled in the art.

In certain aspects, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### B. Protein Recovery

As further detailed hereinafter and presented in the Examples, the instant disclosure further describes and exemplifies particularly suitable processes (methods) for harvesting, clarifying, recovering, purifying and the like fermentation broths in which one or more phosphatase proteins have been produced. Thus, certain embodiments are related to, inter alia, collecting broths at the end of fermentation, harvesting collected broths, recovering one or more phosphatase from a harvested broth (e.g., such as clarifying harvested broths, concentrating clarified broths, purifying clarified broth concentrates, etc.). In certain aspects, purified protein preparations are derived from fermentation broths collected and harvested as described herein.

Certain other aspects of the disclosure provide, inter alia, novel methods for the recovering and optionally purifying recombinantly-produced proteins (such as phosphatases) obtained from a recombinant cells expressing a recombinantly-produced protein (e.g., a recombinant Gram-negative cell, a recombinant Gram-positive cell, a recombinant a plant (e.g., tobacco) cell, etc.). Certain other aspects of the disclosure provide, inter alia, novel methods for the recovery and optional purification of phosphatases obtained from naturally occurring sources.

Thus, in certain aspects, a phosphatase protein preparation is recovered according to the compositions and methods of the disclosure. In other aspects, a phosphatase preparation is recovered and purified according to the methods of the disclosure. As used herein, the terms “purified”, “isolated” or “enriched” with regard to a protein means that the phosphatase is transformed from a less pure state by virtue of separating it from some, or all of, the contaminants with which it is associated. Contaminants include, but are not limited to, microbial cells, metabolites, solvents, chemicals, color, inactive forms of the target phosphatase, aggregates, process aids, inhibitors, fermentation media, cell debris, nucleic acids, proteins other than the target phosphatase protein, host cell proteins, cross-contaminants from the production equipment and the like.

Thus, in the context of a “purified phosphatase” as used herein, purification may be accomplished by any art-recognized separation techniques, including, but not limited to, ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis, or separation on a gradient to remove whole cells, cell debris, impurities, extraneous proteins, or enzymes undesired in the final composition.

It is further possible to then add constituents to a purified or isolated phosphatase composition which provide additional benefits, for example, activating agents, anti-inhibition agents, desirable ions, compounds to control pH or other enzymes or chemicals.

As used herein, phosphatase “purity” is a relative term, and is not meant to be limiting, when used in phrases such as a “recovered phosphatase is of higher purity, the same purity, or lower purity than prior to the recovery process”. For example, the relative “purity” of a protein, before and after a recovery process, may be determined using methods known in the art, including but not limited to, general quantification methods (e.g., Bradford, UV-Vis, activity assays), electrophoretic analysis (SDS-PAGE), analytical HPLC, mass spectrometry, hydrophobic interaction chromatography and the like.

Thus, according to certain aspects, phosphatase protein preparations are recovered from fermentation broths, wherein the recovered phosphatase preparations are of higher purity after performing one or more recovery processes described herein. For example, a fermentation broth (e.g., a whole broth at the end of fermentation) may be subjected to one or more protein recovery processes including, but not limited to, broth conditioning processes, broth clarification processes, protein enrichment and/or protein purification processes (e.g., protein concentration, filtration, precipitation, crystallization, crystal separation, crystal sludge dissolution processes and the like), buffer exchange processes, sterile filtration processes and the like. In certain aspects, the fermentation broth is subjected to a broth treatment (broth conditioning) process to improve subsequent broth handling properties.

In certain embodiments, such as when a Gram-positive host cell has been constructed for intracellular phosphatase expression, a fermentation broth is subjected to a cell lysis process. For example, cell lysis processes include without limitation, enzymatic treatments (e.g., lysozyme, proteinase K treatments), chemical means (e.g., ionic liquids), physical means (e.g., French pressing, ultrasonic), simply holding culture without feeds, and the like. Thus, in certain preferred embodiments, a broth lysis process releases intracellular phosphatase into the (lysed) cell broth.

Thus, as described herein, the methods/processes of the disclosure are not meant to be limiting, as one of skill may readily adapt or modify one or more of the compositions and/or methods disclosed herein for the recovery of specific phosphatase proteins, and/or combinations thereof.

The invention can be further understood by reference to the following examples, which are provided by way of illustration and are not meant to be limiting.

### EXEMPLARY EMBODIMENTS

1. A recombinant Gram-positive host cell comprising a nucleic acid at least about 60% identical to SEQ ID NO:9 or SEQ ID NO:37 or a fragment thereof, wherein the host cell comprises a deletion of one or more endogenous genes encoding a protease.
2. The host cell of embodiment 1, wherein the nucleic acid encodes a polypeptide at least about 80% identical to SEQ ID NO:10 or SEQ ID NO:25 or a functional fragment thereof.
3. The host cell of embodiment 1 or embodiment 2, wherein the nucleic acid encodes a polypeptide at least about 80% identical to SEQ ID NO:26, SEQ ID NO:27 or SEQ ID NO:28 or a functional fragment thereof.
4. The host cell of any one of embodiments 1-3, wherein the host cell has one, two, three, four, five, six, seven, eight, or nine endogenous genes encoding a protease deleted.
5. The host cell of any one of embodiments 1-4, wherein the host cell comprises nine endogenous genes encoding a protease deleted.
6. The host cell of any one of embodiments 1-5, wherein the one or more endogenous genes encoding a protease comprise one or more of aprE, nprE, epr, ispA, bpr, mpr, vpr, wprA and/or nprB.
7. The host cell of any one of embodiments 2-6, wherein the polypeptide comprises one or more disulfide bonds.
8. The host cell of embodiment 7, wherein the polypeptide comprises seven disulfide bonds. 9. The host cell of any one of embodiments 1-8, wherein the nucleic acid is expressed on an extrachromosomal vector.
10. The host cell of any one of embodiments 1-9, wherein the nucleic acid is integrated into the genome of the host cell.
11. The host cell of any one of embodiments 1-10, wherein the host cell is a Bacillus spp. 12. The host cell of embodiment 11, wherein the Bacillus spp. is B. subtilis. 13. The host cell of any one of embodiments 2-12, wherein the polypeptide is secreted from the host cell.
14. A method for producing a recombinant protein comprising culturing the host cell of any one of embodiments 1-13 in a suitable media.
15. The method of embodiment 14, further comprising purifying the recombinant protein.

### EXAMPLES

### Example 1: Expression of Gallaecimonas xiamenensis Apyrase in Bacillus subtilis

A first DNA fragment comprising a (5′) skfA gene flanking region (5′ skfA gene FR, SEQ ID NO: 7) was operably linked to a polynucleotide construct (eg. expression cassette) comprising a (5′) B. subtilis rrnI-p2 promoter region DNA sequence (SEQ ID NO:24) operably linked to a nucleotide sequence of the B. subtilis aprE 5′ untranslated region (5′ UTR) (SEQ ID NO:1) operably linked to a nucleotide sequence encoding the B. subtilis aprE signal sequence (SEQ ID NO: 2), which encodes the aprE signal sequence SEQ ID NO:3, operably linked to a nucleotide sequence codon optimized for Bacillus encoding a mature G. xiamenensis apyrase (CRC22110) gene with the inclusion of the “AGK” peptide at the N-terminus (SEQ ID NO: 9) operably linked to the Bacillus amyloliquefaciens BPN terminator (SEQ ID NO:4) operably linked to a (3′) skfA gene flanking region (3′ skfA gene FR) (SEQ ID NO:8).

A second DNA fragment comprising a (5′) aprE gene flanking region (5′ aprE gene FR) (SEQ ID NO:5) was operably linked to a polynucleotide construct (eg. expression cassette) comprising the nucleotide sequence of B. subtilis rrnI-p2 (SEQ ID NO:24) promoter region operably linked to the nucleotide sequence of B. subtilis aprE 5′ untranslated region (5′ UTR) (SEQ ID NO:1) operably linked to a nucleotide sequence encoding B. subtilis aprE signal sequence (SEQ ID NO:2) operably linked to a nucleotide sequence encoding the mature G. xiamenensis apyrase (SEQ ID NO: 9) gene operably linked to Bacillus amyloliquefaciens BPN terminator (SEQ ID NO:4) operably linked to a (3′) aprE gene flanking region (3′ aprE gene FR) (SEQ ID NO:6).

More particularly, these DNA fragments were assembled using standard molecular biology techniques and were used as templates to develop linear DNA expression cassettes.

A nine protease Bacillus subtilis strain was used for transformation of the two apyrase cassettes derived as described above. The nine proteases deleted in this strain are aprE, nprE, epr, ispA, bpr, mpr, vpr, wprA and nprB.

The Bacillus subtilis strain AP186, a delta nine proteases strain, comprising apyrase expression sequences was constructed by integrating a first and second DNA fragment described above in the genome where the first and second fragments contain the same expression cassette. Secretion the produced apyrase protein was measured using the malachite green method and adenosine triphosphate (ATP) substrate as described above and normalized to the negative control, a non-apyrase expressing Bacillus subtilis host strain AP128 over several timepoints. Bacillus subtilis AP186 expressing the apyrase gene and the negative control strain AP128 (not expressing the apyrase gene) were grown in 24-well MTPs in cultivation medium (enriched semi-defined media based on MOPs buffer, with urea as major nitrogen source, maltodextrin as the main carbon source, supplemented with 3% soytone for robust cell growth) for three (3) days at 37° C., 250 rpm, with 70% humidity in shaking incubator. Clarified and desalted culture supernatants were used to measure (assay) reporter apyrase activity to determine productivity levels, wherein samples were taken after 16.8, 24.8, 40.8, 48 and 65 hour timepoints.

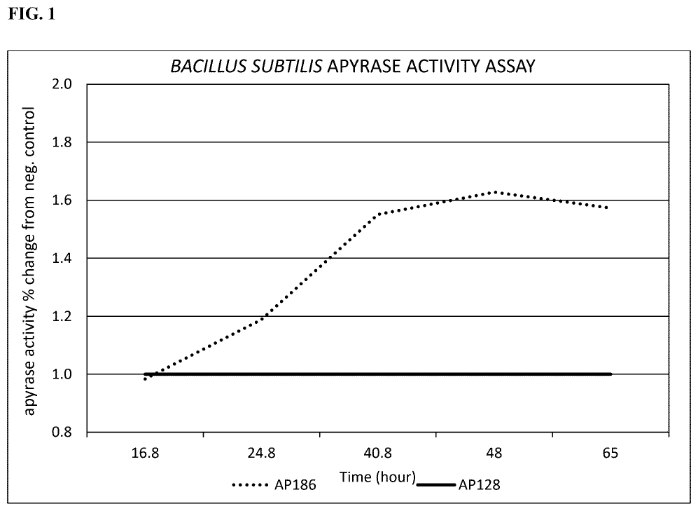

FIG. 1 depicts the measurement of ATPase activity of CRC22110 during a 65 hour time course of cultivation of strains AP186 and AP128. Time dependent ATPase activity was detected from cultures of Bacillus subtilis host strain AP186, as compared to the non-apyrase-expressing, negative control Bacillus subtilis strain AP128.

Clarified culture supernatant from Bacillus subtilis CBS12 strain expressing CRC22110 was used to purify the enzyme. A HiPrep™ Butyl FF column was used as first purification step. The fractions containing the target protein were identified by SDS-PAGE and malachite green assay, using ATP substrate. Active enzyme fractions were pooled and buffer exchanged to 20 mM Tris pH7.5 before loading onto a HiPrep™ Q FF column for subsequent separation step. A NaCl 0-1.0M gradient was used for elution and active fractions identified as mentioned above. Pooled fractions were then run on a Superdex 75 column with 20 mM Tris pH7.5, 150 mM NaCl, 5% Propylene glycol. The active enzyme fractions were identified, pooled and adjusted to 40% glycerol and stored at −20° C. The enzyme purity was assessed by SDS-PAGE and was determined to be >90%.

Purified protein sample was analyzed by a size-exclusion liquid chromatography-electrospray ionization mass spectrometry (SEC-LC-ESI/MS) method as described here. An Orbitrap Eclipse Tribrid mass spectrometer coupled to a Vanquish UPLC (Thermo) with an Acquity BEH-SEC, 200 A HPLC column was used for separation and analysis. The MS parameters were ESI (+) ionization, HESI Probe, 4000V set to Intact Protein/Low pressure mode. Data analysis was performed using the Freestyle (Thermo) program and MS Refiner (Genedata) tool. The deconvoluted mass spectra observed is consistent with the presence of the full length mature CRC22110 polypeptide with 7 Cys-Cys intramolecular bonds. SEQ ID NO:10 corresponds to the full length mature CRC22110 apyrase polypeptide with the N-terminal AGK peptide. Polypeptides consistent with truncations of 2 to 20 amino acids from the C-terminus were also observed. The masses for all these truncated polypeptides match the expected protein with 7 intramolecular disulfide bonds. Sample heterogeneity consistent with the presence of full-length and truncated peptides was also observed using hydrophobic interaction chromatography (HIC) with all protein fractions separated by HIC being enzymatically active on the ATP substrate phosphate release assay.

### Example 2: Expression of Gallaecimonas xiamenensis Apyrase in E. coli

A first DNA fragment (SEQ ID NO: 14) encoding an E. coli codon-optimized mature G. xiamenensis apyrase (SEQ ID NO: 15) was synthesized. This fragment was inserted into an E. coli plasmid pINT (SEQ ID NO:16) comprising of an origin of replication, an antibiotic marker, and a constitutive lacI promoter (SEQ ID NO:20) driving a lacI gene (SEQ ID NO: 19) such that the G. xiamenensis apyrase sequence was operably linked to a T7 RNA polymerase promoter (SEQ ID NO: 22) operably linked to a lacI operator sequence (SEQ ID NO:21). A T7 terminator (SEQ ID NO: 23) was operably linked to the 3′ end of the apyrase. These DNA fragments were assembled using standard molecular biology techniques and were used as a template to construct circular DNA plasmid pINT-CRC2210 containing the apyrase expression cassette.

A second DNA fragment encoding an E. coli codon-optimized mature G. xiamenensis apyrase (SEQ ID NO: 13) was synthesized. This second fragment was inserted into a suitable E. coli plasmid pSCR (SEQ ID NO:17). This second plasmid is comprised of an origin of replication, an antibiotic marker and a constitutive lac/promoter (SEQ ID NO:20) driving a lacI gene (SEQ ID NO: 19), an E. coli pelB signal nucleotide sequence (SEQ ID NO:11) encoding the E. coli pelB signal peptide SEQ ID NO: 12, and a T7 RNA polymerase promoter. The T7 RNA polymerase promoter (SEQ ID NO: 22) was operably linked to a lacI operator sequence (SEQ ID NO:21) operably linked to an E. coli pelB signal sequence. This second DNA fragment was inserted into the pSCR plasmid such that the E. coli pelB signal sequence was operably linked to the G. xiamenensis apyrase which served to direct the mature protein to the periplasm. The 3′ side of the apyrase sequence was operably linked to a T7 terminator (SEQ ID NO: 23). These DNA fragments were assembled using standard molecular biology techniques and were used as a template to construct circular DNA plasmid pSCR-CRC22110 containing an apyrase expression cassette.

E. coli strain AP390 was created by transforming a E. coli plasmid pSCR which does not contain an apyrase fragment into the E. coli strain BL21 (DE3). E. coli strain AP391 was created by transforming the E. coli plasmid pSCR-CRC22110 into E. coli strain BL21 (DE3).

E. coli strain AP392 was created by transforming the E. coli plasmid pINT which does not contain an apyrase fragment in the E. coli strain BL21 (DE3) pLysS which carries pLysS, a compatible plasmid that produces T7 lysozyme, thereby reducing basal expression of target genes and providing even greater stringency. E. coli strain AP393 was created by transforming the E. coli plasmid pINT-CRC22110 in the E. coli strain BL21 (DE3) pLysS.

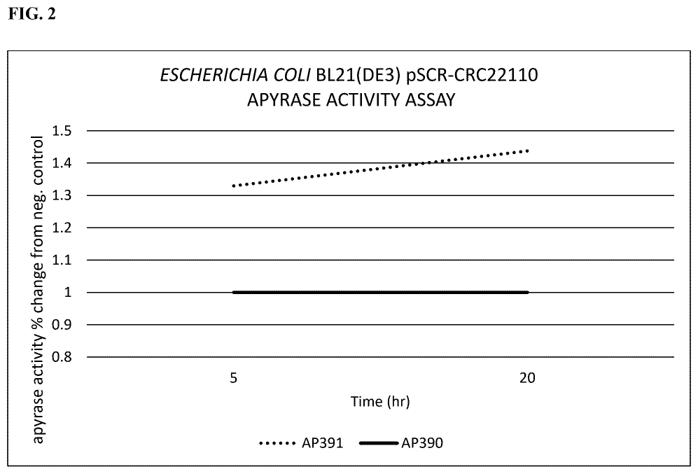

All E. coli strains were cultured in a suitable growth medium using standard molecular biology techniques. Expression of the apyrase protein was induced via addition of 1 mM Isopropyl β-D-1-thiogalactopyranoside to cell cultures. Production of the apyrase protein was measured using the malachite green method and adenosine triphosphate (ATP) substrate as described above and normalized to the appropriate negative control, non-apyrase expressing E. coli strain AP390 over two timepoints. FIG. 2 depicts the measurement of ATPase activity of AP391 containing pSCR-CRC22110 plasmid during a 20 hour time course of cultivation of strain compared to the appropriate negative control AP390 strain containing the control plasmid pSCR. A negligible level of ATPase activity was detected from analysis of AP392 and AP393 samples (and the negative control AP390). The lack of intracellular expression of apyrase by the AP391 strain is consistent with the need to direct the apyrase polypeptide to the periplasm using a signal sequence such as pelB, to enable correct formation of disulfide bonds and expression of an active apyrase enzyme in that specialized environment.

### Example 3. Expression of Truncated Forms of Enzymes of Interest

This Example describes the generation of various C-terminal truncations of the CRC22110 apyrase described in Example 1. Truncated variants were designed to remove residues C-terminal to the final Cysteine which is involved in 1 of the 7 disulfide bonds in the mature enzyme: CRC22110-V1 (SED ID NO: 26), CRC22110-V2 (SED ID NO: 27), and CRC22110-V3 (SED ID NO: 28). The methods of expression of truncation forms of CRC22110 are described below, wherein certain instances an additional 3 residues, AGK, are included at the predicted N-terminus of the mature polypeptide sequence (CRC22110-V1 (SED ID NO: 34), CRC22110-V2 (SED ID NO: 35), and CRC22110-V3 (SED ID NO: 36) correspond to the apyrase proteins expressed with N-terminal AGK). A first DNA fragment comprising a (5′) skfA gene flanking region (5′ skfA gene FR, SEQ ID NO:7) was operably linked to a polynucleotide construct (eg. expression cassette) comprising an upstream (5′) B. subtilis rrnI-p2 promoter region DNA sequence (SEQ ID NO: 24) operably linked to DNA sequence of the B. subtilis aprE 5′ untranslated region (5′ UTR) (SEQ ID NO:5) operably linked to DNA encoding the B. subtilis aprE signal sequence (SEQ ID NO: 2) operably linked to a DNA sequences SEQ ID NO: 31, 32, or 33 encoding the desired C-terminal truncated G. xiamenensis apyrases with the addition of the nucleotide sequences encoding the tripeptide AGK at the N-termini (polypeptide sequences SEQ ID NO: 34, 35 and 36), operably linked to the Bacillus amyloliquefaciens BPN terminator (SEQ ID NO:4) operably linked to a (3′) skfA gene flanking region (3′ skfA gene FR) (SEQ ID NO:8). A second DNA fragment comprising a (5′) amyE gene flanking region (5′ amyE gene FR) (SEQ ID NO:29) was operably linked to a polynucleotide construct (eg. expression cassette) comprising an upstream (5′) B. subtilis rrnI-p2 (SEQ ID NO:24) promoter region DNA sequence operably linked to DNA sequence of the B. subtilis aprE 5′ untranslated region (5′ UTR) (SEQ ID NO:5) operably linked to DNA encoding the B. subtilis aprE signal sequence (SEQ ID NO:2) operably linked to a DNA sequence encoding the desired C-terminal truncated G. xiamenensis apyrases with the addition of the nucleotide sequences encoding the tripeptide AGK at the N-termini (polypeptide sequences SEQ ID NO: 34, 35 and 36), and operably linked to Bacillus amyloliquefaciens BPN terminator (SEQ ID NO:4) operably linked to a (3′) amyE gene flanking region (3′ amyE gene FR) (SEQ ID NO: 30). More particularly, these DNA fragments were assembled using standard molecular biology techniques and were used as templates to develop linear DNA expression cassettes for generation of a 2 copy strain. The Bacillus subtilis strain comprising nine protease deletion, as described in Example 1, was used to integrate the first and second linear DNA expression cassette fragment described above in the genome using standard molecule biology techniques.

### Example 4. Biochemical Evaluation of Truncated Forms of CRC22110 Apyrase

The relative enzyme activity on ATP substrate was determined for truncated forms of the CRC22110 apyrase as described below. Purified samples of CRC22110 (SEQ ID NO:10), CRC22110-V1 (SEQ ID NO:34), CRC22110-V2 (SEQ ID NO:35), and CRC22110-V3 (SEQ ID NO: 36) were prepared and quantified as described below.

Enzyme Isolation: Culture supernatants from B. subtilis fermentations were obtained by filtration and ammonium sulfate and 1M Tris pH8 was added to a final concentration of 1M ammonium sulfate and 20 mM Tris pH8. The sample was centrifuged and filtered. The filtrate was loaded onto 300 ml phenyl Sepharose column on equilibrated in 20 mM Tris and 1M ammonium sulfate pH8. A linear gradient from 0% to 100% 20 mM Tris pH8 was run at 10 ml/min over 300 minutes. Elution fractions were collected and ATPase activity was monitored using ATPase activity assay. The active fractions were pooled, concentrated and buffer exchanged into 20 mM Tris pH8. This pool was loaded onto 25 ml Q Sepharose column that was equilibrated in 20 mM Tris pH8. They were then eluted with step gradient from 50 mM NaCl in 20 mM Tris, followed by 100 mM NaCl, 200 mM NaCl, 400 mM and 500 mM NaCl in 20 mM Tris pH8. The active and pure fractions (>95% by inspection on SDS-PAGE) were pool as purified protein, they were then quantitated.

Protein quantitation by UPLC: Protein quantitation methods: Protein concentration was determined by UPLC (Ultra Performance Liquid Chromatography) and by OD280 densitometry). For UPLC determination, the purified enzyme was diluted in 20 mM Tris pH8 and concentration was determined by separation of protein components using a Zorbax 300 SB-C3 column (Agilent) and running a linear gradient of 0.1% Trifluoroacetic acid in water (Buffer A) and 0.1% Trifluoroacetic acid in Acetonitrile (Buffer B) with detection at 220 nm column on UHPLC. A 10 ul sample was loaded onto the column and the peak area of the diluted sample was determined. The enzyme concentration of the samples was calculated using a standard curve of the purified reference enzyme for example full length CRC22110. The protein concentration was also determined by OD280 measurement. The purified enzyme was diluted in 20 mM Tris pH8, their OD280 was measured in a quartz cuvette. The protein concentration was calculated based on their respective extinction coefficients. The final concentration was calculated based on the average of UPLC method and OD280 determination.

ATPase activity comparison: ATPase activity assay was performed using Bacillus subtilis strains expressing the mature full length CRC22110 apyrase and the truncated variants CRC22110-V1, CRC22110-V2 and CRC22110-V3, which were cultured alongside a non-apyrase expressing Bacillus subtilis host strain CBS12-1 (negative control) in standard soytone MOPS-based media for up to 48.75 hours. Production of the apyrase protein was determined for clarified culture supernatants as described previously using apyrase assay measuring apyrase activity on adenosine triphosphate substrate. Samples of 10 ul of diluted enzyme were added into 0.25 mM ATP in assay buffer to start the reaction. The reaction mix was incubated at 25° C. for 10 mins, then 50 ul of reaction mix was added into 100 ul of QuantiChrom™ Malachite Green reagent (VWR cat #75878) and reaction mixtures were then incubated at 25° C. for 20 mins. The activity values we normalized to the culture biomass measured by absorbance at OD600, and the results are shown on Table 1 as fold change of the negative control. The 3 truncated variants showed similar expression to the full length CRC22110 parent molecule.

TABLE 1
Comparison of ATPase activity for CRC22110 apyrase and C-terminal
truncations thereof with non-apyrase expressing Bacillus host

| Strain | SEQ ID NO | FOLD CHANGE |
|---|---|---|
| CRC22110 | 10 | 1.90 |
| CRC22110-V1 | 34 | 2.05 |
| CRC22110-V2 | 35 | 1.90 |
| CRC22110-V3 | 36 | 1.97 |
| CBS12-1 control | — | 1 |

### Example 5. Disulfide Bond Mapping

This Example describes disulfide bond mapping for CRC22110-V1.

Briefly, a CRC22110-V1 protein sample diluted in equal volume of 2 M freshly made urea solution in 0.1M phosphate buffer (pH=6.9) was incubated in the presence of 1 mM N-ethylmaleimide (final concentration) for 30 min at room temperature to derivatize reduced (free) cysteine residues. Trypsin/LysC mix (Promega, Madison) was then added to the N-ethylmaleimide-treated sample at the enzyme-protein ratio of 1:25 (w/w) and the mixture was incubated at 25° C. overnight.

Chromatographic separation of digest peptides was performed using a Vanquish UPLC coupled to Eclipse Orbitrap Tribrid mass spectrometer (Thermo). Peptides were separated on a Kinetex XB-C18 (150×1 mm, 2.6 uM, 100 A) column eluted at 100 uL/min solvent flow rate with a gradient of 0.1% formic acid in water (Mobile Phase A) and 0.1% formic acid in acetonitrile (Mobile Phase B) at 50° C. The gradient consisted of 5% B for 2 min, increased to 50% B from 2 to 40 min, increased to 100% B from 40 to 45 min, held at to 100% B from 45 to 47 min, then returned to the initial condition of 5% B.

For mass spectrometric (MS) analysis, eluant from the column was introduced into the mass spectrometer using a heated electrospray ionization (HESI) probe operating in a positive mode using a standard application for peptide mapping with MS/MS spectra collected either after HCD or ETD fragmentation of peptides. Collected data were processed by MS Refiner (Genedata) software.

No peptides containing N-ethylmaleimide-modified cysteine residues were detected, suggesting that all cysteine residues in the protein were forming disulfide bonds.

Identification of Cys9-Cys93, Cys181-Cys184, Cys226-Cys242, and Cys345-Cys350 cysteine pairs involved in disulfide bond formation were assessed from MS and MS/MS data; the corresponding peptide pairs and their HCD fragments support those assignments with high conference. No scrambled disulfide bonds were detected. Exact interactions of Cys298, Cys299, Cys305 and Cys312 could not be determined because all four cysteine residues are contained within the same tryptic peptide encompassing amino acid residues 280 through 314 [280-314]. However, match between predicted mass of this peptide with two disulfide bonds and experimentally determined masses for +4 and +3 ions within less than 2.8 ppm, as well as MS/MS fragment pattern, indicates that all four cysteine residues are involved in disulfide bond formation within the same peptide.

Identification of disulfide bond pairs involving residues Cys345-Cys350 and Cys358-Cys365 was based on MS/MS spectra of agglomerate of three disulfide-bond linked peptides ([315-346], [347-359] and [362/363-367]) for which several y ions corresponding to the mass of [353-(C358)-359]=[362/363-(C365)-367] and [356-(C358)-359]=[362/363-(C365)-367] disulfide bond-linked peptides, as well as the ETD fragments corresponding to the mass of [315-346]=[347-359] and [347-359]=[63-367] peptide pairs (“=” means disulfide bond in this notation) was detected. No MS peaks that could be attributed to the disulfide bond arrangement other than described were found.

We claim:

1. A recombinant Gram-positive host cell comprising a nucleic acid at least about 60% identical to SEQ ID NO:9 or SEQ ID NO:37 or a fragment thereof, wherein the host cell comprises a deletion of one or more endogenous genes encoding a protease.

2. The host cell of claim 1, wherein the nucleic acid encodes a polypeptide at least about 80% identical to SEQ ID NO:10 or SEQ ID NO:25 or a functional fragment thereof.

3. The host cell of claim 1 or claim 2, wherein the nucleic acid encodes a polypeptide at least about 80% identical to SEQ ID NO:26, SEQ ID NO:27 or SEQ ID NO:28 or a functional fragment thereof.

4. The host cell of any one of claims 1-3, wherein the host cell has one, two, three, four, five, six, seven, eight, or nine endogenous genes encoding a protease deleted.

5. The host cell of any one of claims 1-4, wherein the host cell comprises nine endogenous genes encoding a protease deleted.

6. The host cell of any one of claims 1-5, wherein the one or more endogenous genes encoding a protease comprise one or more of aprE, nprE, epr, ispA, bpr, mpr, vpr, wprA and/or nprB.

7. The host cell of any one of claims 2-6, wherein the polypeptide comprises one or more disulfide bonds.

8. The host cell of claim 7, wherein the polypeptide comprises seven disulfide bonds.

9. The host cell of any one of claims 1-8, wherein the nucleic acid is expressed on an extrachromosomal vector.

10. The host cell of any one of claims 1-9, wherein the nucleic acid is integrated into the genome of the host cell.

11. The host cell of any one of claims 1-10, wherein the host cell is a Bacillus spp.

12. The host cell of claim 11, wherein the Bacillus spp. is B. subtilis.

13. The host cell of any one of claims 2-12, wherein the polypeptide is secreted from the host cell.

14. A method for producing a recombinant protein comprising culturing the host cell of any one of claims 1-13 in a suitable media.

15. The method of claim 14, further comprising purifying the recombinant protein.


SEQUENCE LISTING (37 sequences)

SEQ ID NO: 1 - DNA, 58 bp, Bacillus subtilis
  source 1..58: mol_type = genomic DNA
acagaatagtcttttaagtaagtctactctgaatttttttaaaaggagagggtaaaga

SEQ ID NO: 2 - DNA, 87 bp, Bacillus subtilis
  source 1..87: mol_type = genomic DNA
gtgagaagcaaaaaattgtggatcagcttgttgtttgcgttaacgttaatctttacgatggcgttcagcaacatgtctgcgcaggct

SEQ ID NO: 3 - protein, 29 aa, Bacillus subtilis
VRSKKLWISLLFALTLIFTMAFSNMSAQA

SEQ ID NO: 4 - DNA, 254 bp, Bacillus amyloliquefaciens
  source 1..254: mol_type = genomic DNA
tctagatacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 5 - DNA, 2982 bp, Bacillus subtilis
  source 1..2982: mol_type = genomic DNA
gcttcaaagcacccgtctcgtcaacgcctgcctcagcaacattgaatttgcagaagaaaaatggcggataaaagactataatatcaacagccacttatccggctttatcaaataagaaaaagacaggcgtttgcctgtcttttcttttatttcttagcagccggcatctctttttgaagctcgtccaaaatggcattcgccccgtctacactgcggcgcagagaccacaccgcacgatccacgtggtatacatgcccgtttttcactgccttcagttttttccaaaggacattcttttcgatcgggcgtttaccgtcggcgtcgaggtcatctgtttttcctgtcatcaggatgatcacatccggatctgttttcagcagctgctccagtgtcattttcatattcacagagtcgccgccattgcttgaatcgctattgcctgacgtactgattgcatatcggtagccgacctgtgttaaaagtctcgatgtaaagaagttttcatccctggccataatggtatcatttgtatttccgatcaaaagcacggactggctgttcgcgctgattttctgctttgtctcgctaagcttttcttcatgcgccgtcagctttttctccatttccttctccttgccgactgcttttgcaatcgtaagcgaagcgtcaattgtatcctgataatcagcatttaaattattaagtgcaatcgtcggcgctatttttttcagctgatcgtacaccttcttatgccgggtcgtgtcagcaataattaaatcgggttttaatgaagcgattttttccatgcttggctgtgagcgagtgccgacagatgtgtagccgtcaattttcttcagcacatccttgttgatcagctgcttcgctttgttgtcatcggcaaccccgacaggcgtaatgccgagatcaagcagtgtatcaataaaacctagctcaagaacaacaacccgcttcggatgctcaggcacatttgtcttccctaaatcatgtgttaccgccactttatgttctttactgttttgattgccgcttgaagacgagcaagcagccgttaagacagaaagaagtaaaactgtaagaataatcagtgtttttttcatatgttccagtctctcctgttggtagtttctatggttaagatgtccaagagtagtataacacggaatgagaatcattatcaccaattatttttaaaatgagaagagaaagttcggcttacaggaaaatcttgtttcgcgacacagcagttcagcagctgatcatcctgtccacaaaaaagcttgcagaaaaataacattctctgcaagctgatcctgttaaagcttcacaatcactcttccttgaatgcgattttgcaaaatatcttttaacgcacccggcgtttcttccaatgatacttccctgtccacgatggtcagcagctgatcaggcttgagatcagaagacatgcgctcccaaacagcggctctgacgtccatcggacaatatactgaatcgattccgagcaggcttactccgcgaagaataaaaggatacacggttgccggaacttctcctccgccggttaagccgctcactgcgacagatccgccgtattgaattttgcttaaaagcgaggcaagctgttttccgccgactggatcaaccgctccctgccattgctgcttggacagcgccttaagcgttccgtcatagacatcttccctgctgattacttcgcttgcaccaagctgtttcaaataatcagccgcctcccggtttccggtacttgccaccacatcataaccccgcttgttcagcatcgataccgcaattccgccgacaccgccggttgctcctgtgactagcacgctgcctttttccggagacagaccgttctgttcaagccgatgcactgataacgccgcagtaaatcccgccgttccgtacaccatcgcttcttttaacgaaagattctgtggcaaaggcaccagccagtcaccaggcaccgaagcgtattcacttaatccgccatcacgtgagacaccgagctcatagcttgtcgcgatcacctcatccccctccgcaaaacgcggatcattggaagagacgaccgtacccgcagcatcaatgcctaaaataagcggatactctctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgc

SEQ ID NO: 6 - DNA, 3089 bp, Bacillus subtilis
  source 1..3089: mol_type = genomic DNA
aggtaagtttttatttgtcagcatcctgatgttccggcgcattctcttctttctccgcatgttgaatccgttccatgatcgacggatggctgcctctgaaaatcttcacaagcaccggaggatcaacctggctcagccccgtcacggccaaatcctgaaacgttttaacagcggcttctctgttctctgtcaactcgatcccatactggtcagccttattctcctgataacgcgagacagcattagaaaaaggcgtaaccgcaaagctcaaaacagaaaacaaaagcaataacagcggaagtgccgcaagatcatgccgcccttctaaatgaaacatgctgcgggttaggcgaaccgtccgcttgtaaagcttatcaatgacataaaatccggcgagcgacacgagcaaatagccagccagaccgatgtaaacgtgcttcatgacataatggcccatttcgtggcccataataaacagaatttctgaatcgtcaagtttgttcagcgtcgtatcccacaatacaatccgtttattggccccaattcctgtaacataggcattcagcgcatttgttttttctgacatgttcacttcatatacatggtcagccggaatattggcttcatctgccagctctaaaattttgctttcaagctctttgtttttcagcggataaaaatcattgtataaaggatcgataatgaccggctgaataaaaaacagaaacagcgaaaacggcactgttaacagccaggcgtataaccaccattttttttcatgccttttgatcagccaataaaaaacgagaacgcaaagcgtaaagattggaaagctgatccaaaagctgataacctgatccttagcccagctggccgttgtctgtgtggaaatgttatagtcaagcgatacttgatagcctatccaatctaaaggcagcgtcaccaatgttgtaatcagcgaaagcacaaacacaaaaccaacggtctgcaaaaaccgaaaaggcacggccgcttcgatccatttcttgattttctttgaaacaccgctgacaagcagaacaaaaaacagaaaccaatcaagtggtaccccgataaaaaataaaaaattcttgacattcgaatactgctcggccactgccaactcagacggcttcatgaaagaagccggatcagcctgcgtccctttcacggcttccggtattgcggaatcacccgacaggaaaaaataccaataaaaaaacagcccgtacagcacgtaagcaagtcctgctgccgcaatccacttgcgcataacgcctcactcctcacatcaacccgttacttctattgtaatcataaattcaaattcttagaaccaagctgtgttccgcacttttccacccttttaagcatggaaaccccgatcgctgggaaaactaacaatgtttggagtgatgcaaatgaaaaaaatagtggcagccatcgtggtaatcggtcttgtgtttatcgcatttttttatctttacagccgatcaggcgatgtgtatcaatcggtagacgcggatttgatcacactgtcttcaagcggccaggaagatatcgagattgaaaaaagacagcacgtcaaagatatgctggatattatgaatcagggaaaacaggtgaagacagaaaaaacatcagcccctgattacgaagggacaatcaagtttcataaagaccggtatgactcattcagactatggattgacggcagccagcaagccgtttttttgaaggatggcacatactacaaattaagcaaaaatgatacaaaggcgctgctaaatattattaaaaaagaagcaaaggattgaaaatgaaaaagcgaagctaaccgcttcgctttttcattttattggggcaaaatatctctcagtgcccgtctgagcattttccccgtcgcatttttcggaatatcgtcaagaaacgtaatggcggcaggccgcttgtattttgccagatgcttttcgcagtgctgcatgatgtcctcctctgttaccccagagcgtttcggcaccacatatccctttaccgcttccccgctttgggggtccggcacgccgatgacaaccgcctccttgacgtccggatggctgtacagcacctcctccacctcccgcggatacacattgtatcctcctacaatgatcatgtcttttttccggtcaacaatgtaaaaatagccgtcctcatcccgtcttgccaagtcccccgtataaagccacccgtcttttaatgcatgctctgtttccatcggcattttataatagcccttcatcacattggggcctttcacgatcaattcgccgacctggtgagcgggcagctcgcgtccgagcggatctacgaccttgttttcgacatgtaagatacttgtcccgatggagcccggctttctgcccctgtcaaacgggttaaagcacgtgacgggtgatgcttccgagagcccgtagccttccaaaatggtaacaccgaatttttcttcaaacgccgtcagcaacgcgactggcatggacgcgcctcccgaaatgcacagccggatcgaagaaaaatcatctttctttccgttttcatgctgaaacaagtagttatacattgtaggcacaccggcaaaaatggtcgcctgctgctgcttaacaagcttaaaaacagatgccggactgaattgaggctcaatcaatacagttgcgccgctcatcagcggtgcattcatacagacggttaaacaaaacacgtgaaacatgggaagagcgcagaccacattgtccctctcatccattcccaaatagcctgcgacatcgttggcattgctgtacaaattctgatgtgtcagcatcgcgcctttcggttttccagtcgttcctgacgtatataaaataaccgcggtatcatcaggtacaggttcttggttttgtttagcggcagatgtcggccgcaatatttttgcaaacgttgtcattttcatcctgacctctgggtccgcagcttccggctcggcctcccccgtctggcataaaatgacgagctcaacctttggcagcgattcatgcatgctctcataaagcggc

SEQ ID NO: 7 - DNA, 3025 bp, Bacillus subtilis
  source 1..3025: mol_type = genomic DNA
gccgaatatgtagtttgtggatcaaaagagggtatgttatctatcaaacaaactcgttcagagttagtcgaatcaggtatagaaggccttaaggatattatcttaaaaataaccggagaaaaagtgaaaagttttcatactgatttaagctcccggacaggtgaacgagtgatggtatttaaattatgtaatgatctagagaaaaatttggaaaagatcttataatctaaagaaaataaatgttttgacggtgtggaatttgttgtgaattgaacataaagaactgccgattcttttgttttacaattatggtatcattgataacttttattcatatcctaagcggacataaagagatgtcaattcatagtacactgtcatgttattcctgtcgaaagatctaacatccgctgttgttatgagcggatgtttttttacatctattttaaatacatgaatttgatgctggtgattagtcacttgaacaatatttgaaaacatccttgtcacctgccgggtttccccaagtgtttagtgacatgattatgagaccaacttcgtaaagcttgctgataaatttaagcggtgaacaaaattgatatgctgtcttcagtttcatttcacggaattagtcagagataaacctttaattaaatatatccaaaaaacggattgacatatcgaaatataacgatataataatcgatatggaaccaattgaagtattcaaagccttatcaaatgaatcaaggctgcaaattttacaatggctgaaggagcccgatcgtcattttgcaccccatgaagggattgatatgaacacaatcggggtatgtgtcagtcaaataacagacaaattgaaaatgacgcaatcgacagcttctcaatatcttaccatccttttaagagccggcctaattaaggcggagcgaatcggaaagtacacgtattataaaagagatgaagaagccattgggaaacttgctgactttcttaaaacagagatataaaaataaacatcaaaagatgtttatttttacaccatacatatcgacatattacgatgtgtttatttttttataataacatatcgataattcgtgatatgtttattaataattaaggagtgaatgttttatgtctaacacttggaaaatttatattttagccattgtcagctttttagttggaacctcagagtacatcatttccggaattttggatcaaattgctcatactctcgggatcactttagctgccgcgggccagcttattaccattttctcacttgtatatgctctttctacacccgtacttatggcgttgacagcaagtatggatagacgcaaattgatgatgtatgccctaggtttgtttgtgttcggtaatgtcctggcttttgtactgcctggttatggatggtttattgcagcgcggatcattatggcgatgggagcaggtgtggttgttgtcaccgcattaacgattgccgctaagattgcatcggaagggaagcagggcagtgccatcgctacggtcgtgatgggatttaccgcttctttaatcattggtgttccgcttggaagaatgatagcagtagcattaggctggaagtctgtatttggagccattgctttgttgggattgatcgcaatggtcgttattttctttactcttccgtatactgaaggggataagcctgtgcctttgcttcaacagcttgctcttttcaaaaaacggaaagtggctatgggattatcaatcactttcttctggctcgggggatattctgttgcttacacttatttgtcaccgtatctcttgaacatctcaggtataaatggcaagctgctcagcggtgttttgcttatatttggaattgccagtttggttggatcgaagtttgggggatatagcaccgataaatggggagtgccctttacactcgttggcgggatgacgttgcatatcgtcacactgattctgctgtcacttgttactcattcctatatcggagtgttggtgattctcatattatggtcgtttgccgcatggtccaccggtccgacacagcaatttcacctggctacaatagaaccggaaatgtcaggtgttttgcttagcatgaatcagtcaatgatgcaattcgccatggcggtcggcgcaggtataggaggggtttttgtggaaaacgtatcattggcctcgattacctgggttggtgcgttaggggttatgattgcaattattgcatcattgctgattttcaattcgcaaccgaaacaagcgctaaaagatatcaatcaataatttcgaaagttctaacatccgctcgttatacaagcgggtgttttttttagcgtagtcagtccatgcatgagtcttccaagggccgacatcgttctgtatgtgtcgaaggtgcactgaagctgaatgagatttcttaaggctttgccgcggcgagctgaagtacagtacgattgccttgatcgaacactggaaacacaagagaacgtaaacctgaacatccgcgtaaacgtcaaggaagttgccacttggggagtaaacacttgcatcatctcgctgaaaggcctagacaatgcggatgacagattcgtattgccggaagtaaacacagcgcttgctctgtttccgttgtcgattgcagctgattgcttactaatgctgccctgcatctcggttgtgatgtcgataagcagcgtaatgaagagtgttactgtggagtaagatgtttaacccctctggatattttagccgaaggggtttttagtatagatatttgatttaaattacaggagacttcattcatttaaccaaagtcattgctttcttaatcactaaagtttatttgaaaataatctcttgatttaatttcctcgaagagattttttgtcaatctattaggcatcagaatttttataacataatggaccgtctttttgacgttttgtttatagaacaagaaaatattcaaaacataagtggaaaattaggggtgagctcc

SEQ ID NO: 8 - DNA, 3184 bp, Bacillus subtilis
  source 1..3184: mol_type = genomic DNA
ggtccttcctataagcaggaaggttttttaatgaaatatttttttattagtaggtaaattcatcaaatgttctgctatgctccaaatgtacacctttccgtaagggcaaagtcaaatgtgaaaaaaaactgttccaaagttaatcagaaatttattttcatattgtatccttcggtatcaagtgaaaggagcatcatatgaaaacattatggaaagtcctcaaaattgtttttgtcagcttggctgctttggttttgcttgtatccgtctcggtatttatttatcaccatttccagctaaataaggaggcggcactgctgaaaggtaaaggcacagtagtcgatgttgacggtaaaaagatgaatgtgtatcaagagggaagcgggaaggatacgtttgtgtttatgtccggttcgggtattgctgcgcctgcttatgaaatgaaggggctgtacagcaagttttcaaaagaaaataagattgctgttgtagatcgggcaggttatggatacagtgaagtgtctcacgatgacagagatattgatacggtattggaacagacgaggaaagcgcttatgaaaagcggaaataagcctccttatattttaatgcctcattcgatatccgggattgaagcgatgtattgggcacagaaatatcctaaggaaatcaaggccattattgcgatggatattggattgccccagcagtacgtcacgtataaattgagcggagttgaccgattgaaagtgagagggttccacctgttaacctcgattggttttcatcggtttataccttccgctgtatataatcctgaggtgattcgacagtcgtttttaactgatgaagaaaaagaaatctataaagccattaactttaagcaattttttaatgcagatatggagcatgagcttttacagtcttaccaaaacggcagcaaatctgtgaatctgcctgcgccaaaggaaactcccgtcttgattttagatgcagtctctgaccaaaatagacattcaaagtatgctatacaaaaccgaaaagactatgaagcgtttgcggctcaattcaatactgccgatataaaggaactgaggggaacacacagtatttatttatatcagcctgatcaaatatataaactgtccatggagtttatgagaaaggttcgctaggatgaagggttatcgtattttaatcgttgaggacgatgtgatgattggtgatttgctgcaaaagattttgcagcgcgagggatatcgtgtgatatggaaaacagatggagcggatgtgctttcggtgattcagaaggtggatttggtcattatggatgtgatgctgccgggtgaagacgggtatcaaatgtctgcaaaaatcaaaaagctggggctgggcattccggttatttttctctcagcccgcaatgacatggacagcaagcttcaaggtttgcagatcggcgaggattatatggtaaagccctttgatccgagagagctgctattaagaatgcggaatatgcttgagcatcattatgggacctttacgcaaatcaaacatttgtatattgatgcggtaacgaaaaaagtgttcaatgaaagcctgcatgatgaggtattatttactgcgattgagcggaaaattttcttttatttatatgaaaatagagacagtatcctgacaaaggaacatttctttgaatatctatggcagctcgaagatagaaacccgaatattgtcaatgtgcatattaaaaaaattagagctaaaatcaatgatcaagcgggtgagatgattgaaaatatatatggagaagggtatcggctgaataccgttgtgaagaaatgaagctcaagacaaaatatcagttgttattgtttacggccgtcattagtgttccgatgctattgctggcggtcagtgttttgatgtcggtgatttatgacagcatgtttaaaccgatgaatcatggtatgccctttcacaggtcgtttgcatacccggcaatgatcgttgtatttttgatatcactcttattgttagcttttttattttcaaagtcgattcattctctgttgcataaaatcaatctattaaatcaaaccattcggcatttggcgagtgatcaaagggtgcccgataaaattgaagtgaagcgtgctgatgaaatcggggaactgatcaagtcggtcaatttgttaattgaacggacgacatatcgtgaactggagctgagacagcaggaggaaatcaaaaaggagcttttgcaaaaactgcggcatgacattaatacacctttaacggctctcaggctgcagttattttatttggaagaccaatgtcatggtcaggctgtattcgaatcattgtatcagcaaatcgaatatatctcggaattaactaatgaattcaatctatattccgctgagacgctggaaagctcttatattgtaaatgaagaagtgcgtctaaacgagctattagaaacagcggtgaaaaagtgggattatttatacagtatgagtgggattgagctgcactataagccggcagatcaagatgtgatatggatgagcaacacgttatggatggaaaggctgtttgataatatttttcaaaatacgttaaggcattcaaaagctaaaaagatggaagtcacgattgaacatggcgatgtttttattcgtgatgacggtattggatttgatcggaatgagagcagtgagggacttgggttaaagattattgaggatacatgcaggctgcttgcgattacttatgagctgcatacaaatgataacggaacggggttcttgttttcaaaagagtgaccccgctgatgtttttctttctcctataaaatttatataatagggagaaagaaatggggtgaacatcatatgtttaaaaaaatcatcaaaacgattaagtacctctcaagcagttctagtgaccgatatcgcagacaccggcattacagcagcagccggcgcagacattatcgcagctacagcagcagttcgggcaaaagaagacactacgaccgttatggcggcagtcatcgttacaaacgccggagctacagcagcagttaatccaaaaaaagttcttgttccgcttttgggacaagggtttttttatgtgaccgattgaatggcccggtttagatcctcgcgaatggaag

SEQ ID NO: 9 - DNA, 1176 bp, synthetic construct
  source 1..1176: mol_type = other DNA
gctggaaaagcagataaaccggcatgccgcgcagtttttgatgcaggctcatcaggaacgagactgtttctgtatgttcaacaagaaggcggctggcaaccgtggccgggcgcaaaactgggcgcacttgcagatccggttcgccaacatcaaggacctaaagcaatggcagcattagcagcagatcttgcaaattctcttgaagcacttcgccaagatggacctacgaatagcgaaggccaacctcgctggcaaggctttgattggcaacaagcatgccgcttacaatctgttagcgttcttgcaacggcaggaatgagacttgcagaacaacaagatccggttgcatctgttgcactgtggcaagaagttcaacaagcactggcaggattactgggccctgcagttgcaatcgatgcacgcacactgacgggctttgaagaaggattttatgcatggttagcagcacgcgcacgccttggacgctctgatatgggaatcgttgaaatgggcggagcatcatctcaagttgcatttccgtgccctgcatgcgatcgcgcagatgatgcagttcaacaagttagacttgaaggccaaacgctggatttttatagctatagctttctgggattaggacaagatgaagcaccgaaagttttaggcgttcctcatgcatgccgctatggagcaggagttaaagatcctaattggcaattaggacaatgccaagcacaacttcctcttacgttaggccgcgcacttaaagatccgtataattttcaaggccaaggccaaggcgggtatcgccaagttccgacggaaaaagcaggagttaaagattggttattaacgggcgcatttcaatataatacacctgatcaattagatagctgctgcttaaatcaaggccaatgctatcaaccgtctacggcatgctttcgcgttgcatatttacctctgtatcttgatagccttggagttgatcctcattctcctgatttagatgtttcatggacactgggagcagttctgtgccgcaatgaacaatgcctggcaccgtctaaacctccgtgccgctggagaaaagcaggctgcctggcaccgagcgttggcgatggacgcccggaaggaagaccgggacaacaaccgggcggcgttcaacatagacaacattaa

SEQ ID NO: 10 - protein, 391 aa, synthetic construct
AGKADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 11 - DNA, 66 bp, Escherichia coli
  source 1..66: mol_type = genomic DNA
atgaaatacctgctgccgaccgctgctgctggtctgctgctcctcgctgcccagccggcgatggcc

SEQ ID NO: 12 - protein, 22 aa, Escherichia coli
MKYLLPTAAAGLLLLAAQPAMA

SEQ ID NO: 13 - DNA, 1167 bp, synthetic construct
  source 1..1167: mol_type = other DNA
gcagataaaccggcatgtcgtgcagtttttgatgcaggtagcagcggcacccgtctgtttctgtatgttcagcaagagggtggttggcagccgtggcctggtgcaaaactgggtgcactggcagatccggttcgtcagcatcagggtccgaaagcaatggcagccctggcagcagatctggcaaatagcctggaagcactgcgtcaggatggtccgaccaatagcgaaggtcagcctcgttggcagggttttgattggcagcaggcctgtcgtctgcagagcgttagcgttctggcaaccgcaggtatgcgtctggcagaacagcaggatccggttgcaagcgttgcactgtggcaagaagttcagcaggcgctggcaggtctgctgggtcctgcagttgcaattgatgcacgtaccctgaccggttttgaagagggtttttatgcatggctggcagcacgtgcccgtctgggtcgtagcgatatgggtattgttgaaatgggtggtgcaagcagccaggttgcatttccgtgtccggcatgtgatcgtgcagatgatgcagtgcagcaggttcgtctggaaggtcagaccctggatttttacagctatagttttcttggtctgggccaagatgaagcaccgaaagttctgggtgttccgcatgcatgtcgttatggtgccggtgttaaagatccgaattggcagctgggtcagtgtcaggcacagctgccgctgacactgggtcgtgcactgaaagatccgtataactttcaaggtcaaggccaaggtggttatcgtcaggttccgaccgaaaaagcaggcgttaaagattggctgctgaccggtgcatttcagtataatacaccggatcagctggatagctgttgtctgaatcagggtcagtgctatcagccgagcaccgcatgttttcgtgttgcatatctgccgctgtatctggatagcctgggtgttgatccgcatagtccggatctggatgttagctggaccctgggtgcagttctgtgtcgtaatgaacagtgtctggcaccgagcaaaccgccttgtcgttggcgtaaagccggttgcctggcaccgtcagttggtgatggtcgtccggaaggccgtccgggacagcagcctggtggtgttcagcatcgtcagcactaa

SEQ ID NO: 14 - DNA, 1170 bp, synthetic construct
  source 1..1170: mol_type = other DNA
atggcagataaaccggcatgtcgtgcagtttttgatgcaggtagcagcggcacccgtctgtttctgtatgttcagcaagagggtggttggcagccgtggcctggtgcaaaactgggtgcactggcagatccggttcgtcagcatcagggtccgaaagcaatggcagccctggcagcagatctggcaaatagcctggaagcactgcgtcaggatggtccgaccaatagcgaaggtcagcctcgttggcagggttttgattggcagcaggcctgtcgtctgcagagcgttagcgttctggcaaccgcaggtatgcgtctggcagaacagcaggatccggttgcaagcgttgcactgtggcaagaagttcagcaggcgctggcaggtctgctgggtcctgcagttgcaattgatgcacgtaccctgaccggttttgaagagggtttttatgcatggctggcagcacgtgcccgtctgggtcgtagcgatatgggtattgttgaaatgggtggtgcaagcagccaggttgcatttccgtgtccggcatgtgatcgtgcagatgatgcagtgcagcaggttcgtctggaaggtcagaccctggatttttacagctatagttttcttggtctgggccaagatgaagcaccgaaagttctgggtgttccgcatgcatgtcgttatggtgccggtgttaaagatccgaattggcagctgggtcagtgtcaggcacagctgccgctgacactgggtcgtgcactgaaagatccgtataactttcaaggtcaaggccaaggtggttatcgtcaggttccgaccgaaaaagcaggcgttaaagattggctgctgaccggtgcatttcagtataatacaccggatcagctggatagctgttgtctgaatcagggtcagtgctatcagccgagcaccgcatgttttcgtgttgcatatctgccgctgtatctggatagcctgggtgttgatccgcatagtccggatctggatgttagctggaccctgggtgcagttctgtgtcgtaatgaacagtgtctggcaccgagcaaaccgccttgtcgttggcgtaaagccggttgcctggcaccgtcagttggtgatggtcgtccggaaggccgtccgggacagcagcctggtggtgttcagcatcgtcagcactaa

SEQ ID NO: 15 - protein, 389 aa, Gallaecimonas xiamenensis
MADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 16 - DNA, 5368 bp, Escherichia coli
  source 1..5368: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 17 - DNA, 5493 bp, Escherichia coli
  source 1..5493: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgaaatacctgctgccgaccgctgctgctggtctgctgctcctcgctgcccagccggcgatggccatggatatcggaattaattcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 18 - DNA, 1080 bp, Escherichia coli
  source 1..1080: mol_type = genomic DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcag

SEQ ID NO: 19 - protein, 360 aa, Escherichia coli
VKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 20 - DNA, 78 bp, Escherichia coli
  source 1..78: mol_type = genomic DNA
gacaccatcgaatggcgcaaaacctttcgcggtatggcatgatagcgcccggaagagagtcaattcagggtggtgaat

SEQ ID NO: 21 - DNA, 25 bp, Escherichia coli
  source 1..25: mol_type = genomic DNA
ggaattgtgagcggataacaattcc

SEQ ID NO: 22 - DNA, 19 bp, T7 bacteriophage
  source 1..19: mol_type = genomic DNA
taatacgactcactatagg

SEQ ID NO: 23 - DNA, 48 bp, T7 bacteriophage
  source 1..48: mol_type = genomic DNA
ctagcataaccccttggggcctctaaacgggtcttgaggggttttttg

SEQ ID NO: 24 - DNA, 91 bp, synthetic construct
  source 1..91: mol_type = other DNA
gctgataaacagctgacatcaactaaaagtttcattaaatactttgaaaaaagttgttgacttaaaagaagctaaatgttatagtaattgt

SEQ ID NO: 25 - protein, 388 aa, Gallaecimonas xiamenensis
ADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVGDGRPEGRPGQQPGGVQHRQH

SEQ ID NO: 26 - protein, 364 aa, synthetic construct
ADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLA

SEQ ID NO: 27 - protein, 368 aa, synthetic construct
ADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVG

SEQ ID NO: 28 - protein, 367 aa, synthetic construct
ADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSV

SEQ ID NO: 29 - DNA, 1962 bp, Bacillus subtilis
  source 1..1962: mol_type = other DNA
gttcagctcagtgatacctgcgatccctccgcgattcacgacagccggcacaccgatgtacacgtcatctgcaccgtattgcccgtccaaatatgtgctgacagttaatatgctgttttcattatgaagaatggcttttgtaatgcgagcaagactcatcgcaaccccataataagtcgcgccttttttctcaatgatatggtaagctgcgtttttcacatcatctacaatttggtccagctcctcttgtttgtacgcatcgtttttctcaacgagttcactgaccggcacaccgccgacattcgcgtggctccaaacaggaagctctgtgtcgccgtgctctccgataatatgcgcgtgtacgttttgaggcgctgcgccaaagtattcgctcagcatgaaacggaatctcgcagaatcaagtgttgtgccgcttccaatcacccgctcttttggcaggccgctgaatttccatgttgcgtaagtcaggatatcaaccggatttgtcgcgactaagaaaatgccgtcaaatccgctcgccatgacttcactaacgatgcctttgaaaatcttcaagttcttttctactaattcaaggcgtgtctcaccaggtttttggtttgctccggcgcaaatgcagacaatatcagcatccttgcagtcttcatatgttccgtaagatgttttgaccggttgtagcccaaacgcctttccgtggggtaaatccatcacatcgcccattgctttttctttatttacatcaatgaccacaagctcatctgtgatcccttggttaattaacgcaaatgcataactgcttccaacaaaacccgctccgattaaagctactttatttacatgtttgttcatcattaatcatccttgcagggtatgtttctctttgatgtctttttgtttgtgaagtatttcacatttatattgtgcaacacttcacaaacttttgcaagagaaaagttttgtctgatttatgaacaaaaaagaaaccatcattgatggtttctttcggtaagtcccgtctagccttgccctcaatggggaagagaaccgcttaagcccgagtcattatataaaccatttagcacgtaatcaaagccaggctgattctgaccgggcacttgggcgctgccattattaaaaatcacttttgcgttggttgtatccgtgtccgcaggcagcgtcagcgtgtaaattccgtctgcatttttagtcattggttttccaggccaagatccggtcaattcaattactcggctcccatcatgtttatagatataagcatttacctggctccaatgattcggattttgatagccgatggttttggccgacgctggatctcttttaacaaaactgtatttctcggtcctcgttacaccatcactgttcgttccttttaacatgatggtgtatgttttgccaaattggatctccttttccgattgtgaattgatctccatccttaaacgcctgtcgtctggtccattattgatttgataaacggcttttgttgtattcgcatctgcacgcaaggtaatcgtcagttgatcattgaaagaatgtgttacacctgttttgtaattctcaaggaaaacatgaggcgcttttgcaatatcatcaggataaagcacagctacagacctggcattgatcgtgcctgtcagtttaccatcgttcacttgaaatgaacccgctccagctttattgtcatacctgccatcaggcaattttgttgccgtattgatagagacagaggatgaacctgcatttgccagcacaacgccatgtgagccgcgctgattcataaatatctggttgtttccattcgggttcgagagttcctcaggctgtccagccatcacattgtgaaatctattgaccgcagtgatagcctgatcttcaaataaagcactcccgcgatcgcctatttggcttttccccggg

SEQ ID NO: 30 - DNA, 2111 bp, Bacillus subtilis
  source 1..2111: mol_type = other DNA
tcttgacactccttatttgattttttgaagacttacttcggagtcaaaaatccctcttacttcattcttccgcttcctcctttcaaaccgatgtgaagactggagaattttgttaacgcttacatttaaaattatcacaatcactctatcaaaacaacttggcagagtgaatacaaatcaatgttcctataaaaaaagctgcccgaaactgtgaacgcttccctcgctttccaaacaaaaaagatgatttcttttttatttcttcttttaccgctcttctgtaagccagcttcacaatcacataccgaagcagaaacagaacgccgccgcagacgataaaaagaagaatcattttcagccaagcgaagaatgtatggggaatccccctgtcgtacatcgtaagcaccactaccgcaaaggcgcaagcaattgattctattcgctgctgcttcacccgtttttgcagttcttctttggtgtctattctcggcatgagcattccggatgccgcagccctgatgcagtaatataagataatgatgaacaaaaacacaaggcttgcagaccattctttaaacggaaattcaaacataatgccgcggatgatcacatcaggcagagcaactgctcccgctatcatcccattctccgaccagattttttgttctttttcttttactctcttatttttcccattcacgcctgaaagtctattacgcagattattcatgaccttcctcccgcacttttacgaatggcttatggcttacatgttacaataacagcctgcgcttctgcaatgaataactcaaaagcccgccgtacaagccggcgggcagcaatcgttaaaacggcccccaattgatcatgactccgatcacgataaagacaagcccgatcaagaaccaaatcagagcaagcggcaccataaaccgcagccacttgacatacggaataccgctggcggcaagcaccgccatgagaacgccggatgtcgggttcacacagttgacgaccccttctccaagcataaccgcttcaaccgcaacctgtctcgtgattcccatcaaatcagcgagcggcgccaggattggaataaatacaacggcttcgccagaacctgaagagatgagaaaatgaagcagcgcactggcgatatacatgccgattgccccagcaatcgggctgaatccatccaaaagtgaagccaaagcattgacgacagtatcgagaagctttccattttcaaggataacggaaatgcttcgtgccatcccgacaatcagcgccccgtatacaagactttggcagcccgtaatgaaggttttggcaatatcgttcgccgcaagcccgcctattaaaccggcaaggacagaaataaaaataaatgtcgcagccatttgggaatctgaccagccaagcttcaaagcgccgtataaaaatccgacaagtgagagtcccgccacagccaaaatcagcttatggcgaacggtaaacggcactgactgatcttcttctttaccggcttcgcccattcctgccgcagggaaccaatctgttcctaacacactggcatcttttgatttttttaattttctcgtatacaaatagatataaataatagaagacagcaaaaagcatatgtatatcacaactcgcaggccgattcctgagaaaagagggagctccgcgattgattgtgataaaccgagcggtgacggtgataatatggtggagttaaatccagcgtagcagccgatgtatataacagcggctcccgcgactgcgtcccattttaaggatcgtgccacaatgaggccgatggggataaaaccgataactgaattcacgacaatcccggttgttccgagaatggagaacaatgcgccgacaatacaaataaataataattgctttgtgcggaatttgctgatgacattgtaaatcaggccattgatggcacccgttttttctagaatagcaatggtgccgcctgtgaataaaatcagaaagatgatgggtgctgatccaaccattccatcctgtagagaagtaaaaaagctgatcaatctgaccggagactgttcaattgaatgatagcttcccggaac

SEQ ID NO: 31 - DNA, 1104 bp, synthetic construct
  source 1..1104: mol_type = other DNA
gctggaaaagcagataaaccggcatgccgcgcagtttttgatgcaggctcatcaggaacgagactgtttctgtatgttcaacaagaaggcggctggcaaccgtggccgggcgcaaaactgggcgcacttgcagatccggttcgccaacatcaaggacctaaagcaatggcagcattagcagcagatcttgcaaattctcttgaagcacttcgccaagatggacctacgaatagcgaaggccaacctcgctggcaaggctttgattggcaacaagcatgccgcttacaatctgttagcgttcttgcaacggcaggaatgagacttgcagaacaacaagatccggttgcatctgttgcactgtggcaagaagttcaacaagcactggcaggattactgggccctgcagttgcaatcgatgcacgcacactgacgggctttgaagaaggattttatgcatggttagcagcacgcgcacgccttggacgctctgatatgggaatcgttgaaatgggcggagcatcatctcaagttgcatttccgtgccctgcatgcgatcgcgcagatgatgcagttcaacaagttagacttgaaggccaaacgctggatttttatagctatagctttctgggattaggacaagatgaagcaccgaaagttttaggcgttcctcatgcatgccgctatggagcaggagttaaagatcctaattggcaattaggacaatgccaagcacaacttcctcttacgttaggccgcgcacttaaagatccgtataattttcaaggccaaggccaaggcgggtatcgccaagttccgacggaaaaagcaggagttaaagattggttattaacgggcgcatttcaatataatacacctgatcaattagatagctgctgcttaaatcaaggccaatgctatcaaccgtctacggcatgctttcgcgttgcatatttacctctgtatcttgatagccttggagttgatcctcattctcctgatttagatgtttcatggacactgggagcagttctgtgccgcaatgaacaatgcctggcaccgtctaaacctccgtgccgctggagaaaagcaggctgcctggcataa

SEQ ID NO: 32 - DNA, 1116 bp, synthetic construct
  source 1..1116: mol_type = other DNA
gctggaaaagcagataaaccggcatgccgcgcagtttttgatgcaggctcatcaggaacgagactgtttctgtatgttcaacaagaaggcggctggcaaccgtggccgggcgcaaaactgggcgcacttgcagatccggttcgccaacatcaaggacctaaagcaatggcagcattagcagcagatcttgcaaattctcttgaagcacttcgccaagatggacctacgaatagcgaaggccaacctcgctggcaaggctttgattggcaacaagcatgccgcttacaatctgttagcgttcttgcaacggcaggaatgagacttgcagaacaacaagatccggttgcatctgttgcactgtggcaagaagttcaacaagcactggcaggattactgggccctgcagttgcaatcgatgcacgcacactgacgggctttgaagaaggattttatgcatggttagcagcacgcgcacgccttggacgctctgatatgggaatcgttgaaatgggcggagcatcatctcaagttgcatttccgtgccctgcatgcgatcgcgcagatgatgcagttcaacaagttagacttgaaggccaaacgctggatttttatagctatagctttctgggattaggacaagatgaagcaccgaaagttttaggcgttcctcatgcatgccgctatggagcaggagttaaagatcctaattggcaattaggacaatgccaagcacaacttcctcttacgttaggccgcgcacttaaagatccgtataattttcaaggccaaggccaaggcgggtatcgccaagttccgacggaaaaagcaggagttaaagattggttattaacgggcgcatttcaatataatacacctgatcaattagatagctgctgcttaaatcaaggccaatgctatcaaccgtctacggcatgctttcgcgttgcatatttacctctgtatcttgatagccttggagttgatcctcattctcctgatttagatgtttcatggacactgggagcagttctgtgccgcaatgaacaatgcctggcaccgtctaaacctccgtgccgctggagaaaagcaggctgcctggcaccgagcgttggctaa

SEQ ID NO: 33 - DNA, 1113 bp, synthetic construct
  source 1..1113: mol_type = other DNA
gctggaaaagcagataaaccggcatgccgcgcagtttttgatgcaggctcatcaggaacgagactgtttctgtatgttcaacaagaaggcggctggcaaccgtggccgggcgcaaaactgggcgcacttgcagatccggttcgccaacatcaaggacctaaagcaatggcagcattagcagcagatcttgcaaattctcttgaagcacttcgccaagatggacctacgaatagcgaaggccaacctcgctggcaaggctttgattggcaacaagcatgccgcttacaatctgttagcgttcttgcaacggcaggaatgagacttgcagaacaacaagatccggttgcatctgttgcactgtggcaagaagttcaacaagcactggcaggattactgggccctgcagttgcaatcgatgcacgcacactgacgggctttgaagaaggattttatgcatggttagcagcacgcgcacgccttggacgctctgatatgggaatcgttgaaatgggcggagcatcatctcaagttgcatttccgtgccctgcatgcgatcgcgcagatgatgcagttcaacaagttagacttgaaggccaaacgctggatttttatagctatagctttctgggattaggacaagatgaagcaccgaaagttttaggcgttcctcatgcatgccgctatggagcaggagttaaagatcctaattggcaattaggacaatgccaagcacaacttcctcttacgttaggccgcgcacttaaagatccgtataattttcaaggccaaggccaaggcgggtatcgccaagttccgacggaaaaagcaggagttaaagattggttattaacgggcgcatttcaatataatacacctgatcaattagatagctgctgcttaaatcaaggccaatgctatcaaccgtctacggcatgctttcgcgttgcatatttacctctgtatcttgatagccttggagttgatcctcattctcctgatttagatgtttcatggacactgggagcagttctgtgccgcaatgaacaatgcctggcaccgtctaaacctccgtgccgctggagaaaagcaggctgcctggcaccgagcgtttaa

SEQ ID NO: 34 - protein, 367 aa, synthetic construct
AGKADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLA

SEQ ID NO: 35 - protein, 371 aa, synthetic construct
AGKADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSVG

SEQ ID NO: 36 - protein, 370 aa, synthetic construct
AGKADKPACRAVFDAGSSGTRLFLYVQQEGGWQPWPGAKLGALADPVRQHQGPKAMAALAADLANSLEALRQDGPTNSEGQPRWQGFDWQQACRLQSVSVLATAGMRLAEQQDPVASVALWQEVQQALAGLLGPAVAIDARTLTGFEEGFYAWLAARARLGRSDMGIVEMGGASSQVAFPCPACDRADDAVQQVRLEGQTLDFYSYSFLGLGQDEAPKVLGVPHACRYGAGVKDPNWQLGQCQAQLPLTLGRALKDPYNFQGQGQGGYRQVPTEKAGVKDWLLTGAFQYNTPDQLDSCCLNQGQCYQPSTACFRVAYLPLYLDSLGVDPHSPDLDVSWTLGAVLCRNEQCLAPSKPPCRWRKAGCLAPSV

SEQ ID NO: 37 - DNA, 1167 bp, synthetic construct
  source 1..1167: mol_type = other DNA
gcagataaaccggcatgccgcgcagtttttgatgcaggctcatcaggaacgagactgtttctgtatgttcaacaagaaggcggctggcaaccgtggccgggcgcaaaactgggcgcacttgcagatccggttcgccaacatcaaggacctaaagcaatggcagcattagcagcagatcttgcaaattctcttgaagcacttcgccaagatggacctacgaatagcgaaggccaacctcgctggcaaggctttgattggcaacaagcatgccgcttacaatctgttagcgttcttgcaacggcaggaatgagacttgcagaacaacaagatccggttgcatctgttgcactgtggcaagaagttcaacaagcactggcaggattactgggccctgcagttgcaatcgatgcacgcacactgacgggctttgaagaaggattttatgcatggttagcagcacgcgcacgccttggacgctctgatatgggaatcgttgaaatgggcggagcatcatctcaagttgcatttccgtgccctgcatgcgatcgcgcagatgatgcagttcaacaagttagacttgaaggccaaacgctggatttttatagctatagctttctgggattaggacaagatgaagcaccgaaagttttaggcgttcctcatgcatgccgctatggagcaggagttaaagatcctaattggcaattaggacaatgccaagcacaacttcctcttacgttaggccgcgcacttaaagatccgtataattttcaaggccaaggccaaggcgggtatcgccaagttccgacggaaaaagcaggagttaaagattggttattaacgggcgcatttcaatataatacacctgatcaattagatagctgctgcttaaatcaaggccaatgctatcaaccgtctacggcatgctttcgcgttgcatatttacctctgtatcttgatagccttggagttgatcctcattctcctgatttagatgtttcatggacactgggagcagttctgtgccgcaatgaacaatgcctggcaccgtctaaacctccgtgccgctggagaaaagcaggctgcctggcaccgagcgttggcgatggacgcccggaaggaagaccgggacaacaaccgggcggcgttcaacatagacaacattaa